(19) United States
(12) Patent Application Publication
(10) Pub. No.: US 2021/0245201 A1
(43) Pub. Date: Aug. 12, 2021

(54) A SYSTEM, METHOD AND COMPUTER PRODUCT FOR REAL TIME SORTING OF PLANTS

(71) Applicant: H.T.B AGRI LTD., Ganei Am (IL)

(72) Inventors: Shani TOLEDANO, Givatayim (IL); Gideon BARAK, Hod Hasharon (IL)

(21) Appl. No.: 17/252,005

(22) PCT Filed: Jun. 12, 2019
(86) PCT No.: PCT/IL19/50666

§ 371 Date: Dec. 14, 2020

Related U.S. Application Data
(60) Provisional application No. 62/683,851, filed on Jun. 12, 2018.

(51) Int. Cl. B07C 5/34; G05B 19/406; G06N 20/00

(52) U.S. Cl. CPC B07C 5/34; G05B 19/406; G05B 2219/49219; G05B 2219/37591; G06N 20/00

(57) ABSTRACT

A method including receiving a sequence of thermal data of a plant, wherein said sequence is sampled at least one location of said tissue while said tissue is being thermally disturbed, processing said thermal data to derive thermal values associated with each of said tissue locations, deriving at least one thermal variable at least one location on said plant, based, at least in part, on said processing, calculating a variance value of all said thermal variables associated with each of said locations and determining a state of said plant based on at least one location at which said variance value exceeds a predetermined threshold. The disclosure also includes a system and computer product for real time sorting of plants.

### CROSS REFERENCE TO RELATED APPLICATIONS

This application claims the benefit of priority of U.S. Provisional Patent Application No. 62/683,851 filed on Jun. 12, 2018, entitled “A SYSTEM, METHOD AND COMPUTER PRODUCT FOR REAL TIME SORTING OF AGRICULTURAL PRODUCTS”.

This application is related to International Patent Application No. PCT/IL2015/050392 published as PCT Publication No. WO2015/159284, entitled “A DEVICE AND METHOD FOR CANCER DETECTION, DIAGNOSIS AND TREATMENT GUIDANCE USING ACTIVE THERMAL IMAGING” to the same inventor. The contents of the above applications are all incorporated by reference as if fully set forth herein in their entirety.

### FIELD OF THE INVENTION

The present invention, in some embodiments thereof, relates to thermal systems and methods and more particularly, but not exclusively, to thermal systems and methods for real-time sorting plants.

### BACKGROUND

Thermography is a field in which thermal radiation such as Infra-Red radiation emitted from an object is detected by a sensor (e.g., thermographic camera) that converts the sensed thermal radiation into an image (thermogram). A thermogram allows to observe differences in the thermal radiation emitted from various areas over the imaged object.

Thermal radiation emitted from an object without external thermal intervention—passive thermography—can be higher or lower than the background thermal radiation emitted. Passive thermography has many applications such as surveillance of people against a background and medical diagnosis (specifically thermology).

Unlike in passive thermography, an energy source may actively heat an object—active thermography—to produce a thermal contrast between the object and the background. Active thermography is used in cases in which the inspected object is in equilibrium with the surroundings.

Until recently, plants were sorted by hand. Modern sorting systems commonly include color white image cameras and computer software and hardware to sort or grade plants based on color, weight, size, and internal and external quality.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

According to an aspect of some embodiments of the invention there is provided a method including receiving a sequence of thermal data of a plant the sequence is sampled at least one location of the tissue while the tissue is being thermally disturbed, processing the thermal data to derive thermal values associated with each of the tissue locations, deriving at least one thermal variable at least one location on the plant, based, at least in part, on the processing, calculating a variance value of all the thermal variables associated with each of the locations, and determining a state of the plant based on at least one location at which the variance value exceeds a predetermined threshold.

In accordance with some embodiments, the deriving includes calculating a set of thermal features of each of the tissue locations based, at least in part, on the at least one thermal variable. In some embodiments, the thermal data is received from at least one of thermal imaging, infrared (IR) sensor, mercury thermometer, resistance thermometer, thermistor, thermocouple, semiconductor-based temperature sensor, pyrometer, gas thermometer, laser thermometer and ultrasound. In accordance with some embodiments, the thermal data is received by thermal imaging, and wherein the location includes a pixel or a voxel of an image. In accordance with some embodiments, the at least one thermal variable further includes at least one of ambient temperature and a heat source temperature.

In accordance with some embodiments, the thermal disturbing includes at least one of: actively effecting a change in temperature in at least a portion of the tissue from an initial temperature to an end temperature, actively effecting a change in temperature in at least a portion of the tissue for a specified period of time, passively allowing a change in temperature in at least a portion of the tissue from an initial temperature to an end temperature, and passively allowing a change in temperature in at least a portion of the tissue for a specified period of time. In accordance with some embodiments, the method includes extracting a set of features based on at least some of the thermal data and thermal variables the features are selected from groups of features including: features representing various derivative values of the variables, features representing noise in the variables, features based on decay equations, features based on Fourier series and correlative features based on a variance of the features.

In accordance with some embodiments, the method further includes determining a state of the plant associated with each of the locations, based, at least in part, on correlating the at least one thermal variable with predefined values of the thermal variables associated with a plurality of plant states and the correlating further includes correlating the features. In accordance with some embodiments, the deriving, calculating, and determining is performed by a machine learning classifier trained, at a training stage, on a training set including a) a plurality of thermal data sequences, each sampled at least one location of a tissue, while the tissue is being thermally disturbed, and b) labels associated with a state or type of the at least one location. In accordance with some embodiments, further including applying, at an inference stage, the trained machine learning classifier to at least one target thermal data sequence sampled at a location of a tissue, while the tissue is being thermally disturbed, to determine a state or type of the tissue location.

According to an aspect of some embodiments of the invention there is provided a computer program product including a non-transitory computer-readable storage medium having program code embodied therewith, the program code executable by at least one hardware processor to receive a sequence of thermal data of a plant the sequence is sampled at least one location of the tissue while the tissue is being thermally disturbed, process the thermal data to derive thermal values associated with each of the tissue locations, derive at least one thermal variable at least one location on the plant, based, at least in part, on the processing, calculate a variance value of all the thermal variables associated with each of the locations, and determine a state of the plant based on at least one location at which the variance value exceeds a predetermined threshold.

In accordance with some embodiments, the at least one thermal variable indicates a state of the plant. In some embodiments, the thermal data is received from at least one of thermal imaging, infrared (IR) sensor, mercury thermometer, resistance thermometer, thermistor, thermocouple, semiconductor-based temperature sensor, pyrometer, gas thermometer, laser thermometer and ultrasound. In accordance with some embodiments, the thermal data is received by thermal imaging and wherein the location includes a pixel or a voxel. In some embodiments, the thermally disturbed tissue includes actively or passively effecting a change in temperature on at least a portion of the tissue from an initial temperature to an end temperature.

In accordance with some embodiments, the thermally disturbed tissue includes effecting a change in temperature on at least a portion of the tissue over at least one predetermined period of time. In some embodiments, the at least one tissue-related thermal variable includes at least one intrinsic plant thermal parameter affecting thermal behavior of the plant cell. In accordance with some embodiments, the program is configured to calculate a set of features based on at least some of the thermal data and thermal variables. In some embodiments, the features are selected from groups of features including features representing various derivative values of the variables, features representing noise in the variables, features based on decay equations, features based on Fourier series and correlative features based on a variance of the features.

In some embodiments, the determining the state of the plant at the location is further based on the locations having a corresponding set of features. In some embodiments, the deriving includes calculating a set of thermal features of each of the tissue locations based, at least in part, on the at least one thermal variable.

According to an aspect of some embodiments of the invention there is provided a system, including: a thermal sensor configured to sample a sequence of thermal data from at least one location on tissue while the tissue is being thermally disturbed, and a processor configured to: receive a sequence of thermal data of a plant the sequence is sampled at least one location of the tissue while the tissue is being thermally disturbed, process the thermal data to derive thermal values associated with each of the tissue locations, derive at least one thermal variable at least one location on the plant, based, at least in part, on the processing, calculate a variance value of all the thermal variables associated with each of the locations, and determine a state of the plant based on at least one location at which the variance value exceeds a predetermined threshold.

In some embodiments, the system includes a heating or cooling source directed at least the surface of the plant and configured to actively heat or cool the tissue. In some embodiments, the at least one thermal variable indicates a state or type of the plant. In some embodiments, the thermal data is received from at least one of thermal imaging, infrared (IR) sensor, mercury thermometer, resistance thermometer, thermistor, thermocouple, semiconductor-based temperature sensor, pyrometer, gas thermometer, laser thermometer and ultrasound. In some embodiments, the thermal data is received by thermal imaging and wherein the location includes a pixel or a voxel.

In some embodiments, the thermally disturbed plant includes actively or passively effecting a change in temperature on at least a portion of the tissue from an initial temperature to an end temperature. In some embodiments, the thermally disturbed tissue includes effecting a change in temperature on at least a portion of the plant over at least one predetermined period of time. In some embodiments, the at least one plant-related thermal variable includes at least one intrinsic plant thermal parameter affecting thermal behavior of the plant cell. In some embodiments, the system includes calculating a set of features based on at least some of the thermal data and thermal variables. In some embodiments, the features are selected from groups of features including features representing various derivative values of the variables, features representing noise in the variables, features based on decay equations, features based on Fourier series and correlative features based on a variance of the features.

In some embodiments, the system processor is configured to determine a state of the plant at the location is further based on the locations having a corresponding set of features.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1. is a simplified diagram of a thermal imaging system for real-time plants in accordance with some embodiments of the invention; FIG. 2 is a simplified diagram of a thermal imaging system for real-time plants in accordance with some embodiments of the invention;

FIG. 3 is a simplified diagram of a thermal imaging system for real-time plants in accordance with some embodiments of the invention;

FIG. 4 is a simplified diagram of a thermal imaging system for real-time plants in accordance with some embodiments of the invention;

FIGS. 5A and 5B are graphs of thermal curves associated with biothermal behavior of a heated plant in accordance with some embodiments of the invention;

FIG. 6 is a graph of a thermal curve associated with biothermal behavior of a heated plant in accordance with some embodiments of the invention;

FIG. 7 is a graph of thermal curves associated with biothermal behavior of a heated plant in accordance with some embodiments of the invention;

FIGS. 8A, 8B and 8C are graph analyses of peak temperature points in accordance with some embodiments of the invention;

FIG. 9 is an exemplary simplified flow chart illustrating operation of thermal imaging system processor in accordance with some embodiments of the invention;

FIGS. 10A, 10B and 10C are exemplary screen thermal images of potato tubers on a moving or paused sorting path in accordance with some embodiments of the invention;

FIGS. 11A, 11B and 11C are thermal images comparing a healthy and an unhealthy potato in accordance with some embodiments of the invention;

FIG. 12A is a planar view simplified illustration of heat distribution over a portion of an agricultural product; and

FIG. 12B, is a thermal graph of an plant thermal behavior within the portion of the plant in accordance with some embodiments of the invention;

FIG. 13 is a planar view simplified illustration of heat distribution over a portion of an plant in accordance with some embodiments of the invention;

FIG. 14 is an exemplary simplified flow chart illustrating operation of thermal imaging system processor in accordance with some embodiments of the invention;

FIGS. 15A and 15B are graphs of thermal curves associated with biothermal behavior of heated plant in accordance with some embodiments of the invention;

FIG. 16 is a graph of a thermal curve associated with biothermal behavior of heated plant in accordance with some embodiments of the invention; and

FIGS. 17A and 17B are sectional view simplified illustrations of heat distribution inside a portion of an plant in accordance with some embodiments of the invention.

### DETAILED DESCRIPTION

The present invention, in some embodiments thereof, relates to thermal imaging systems and methods and more particularly, but not exclusively, to thermal imaging systems and methods for real-time sorting of plants.

According to an aspect of some embodiments of the present invention there is provided a method for real-time sorting of plants. The term “plant” as used herein refers to any known type of plant including products of plant growth such as fruits, vegetables, seeds and flowers.

Though plant thermal data can be sampled by many sensing devices e.g., infrared (IR) sensors, mercury thermometers, resistance thermometers, thermistors, thermocouples, semiconductor-based temperature sensors, pyrometers, gas thermometers, laser thermometers and ultrasound, for the purpose of clarity and simplicity, by way of example but not by way of limitation, hereafter, determination of a state of one or more locations on a plant is demonstrated based on thermal properties calculated from data received by thermal imaging.

In some embodiments, the method includes real-time sorting of plants based on a state of at least a portion of the agricultural product. In some embodiments, the method includes real-time sorting of the plants. plants

According to an aspect of some embodiments of the present invention, there is provided a method for in-situ thermal imaging identification of a state of health of one or more plant tissue regions based, at least in part, on thermal properties calculated from one or more thermal images. In some embodiments, the method comprises in-situ thermal imaging determination of a state of one or more plant tissue regions based, at least in part, on thermal properties calculated from the thermal imaging.

According to an aspect in accordance with some embodiments of the invention, there is provided a method for differentiating between healthy and unhealthy plant tissues. In some embodiments, the differentiation is determined in accordance with plant tissue states in situ, based, at least in part, on a change in temperature of the plant tissue. For example, effecting a change in temperature (e.g., heating) of at least a surface of at least a portion of an plant tissue, from a base temperature over a predetermined first period of time e.g., t0 to t1 followed by allowing the temperature of the plant tissue to passively return (e.g., cool) to the base temperature, over a second period of time e.g., t1 to t2. The term “unhealthy state” as used herein refers to any state of a plant tissue rendering the plant unsuitable for marketing, for example, diseased and/or blemished plants.

In some embodiments, during the combined first and second periods of time t0 to t2, a sequence of thermal images, e.g., a video stream, of at least the surface of the plant tissue is obtained using one or more suitable thermal imaging devices, e.g., an infrared (IR), near infrared (NIR), short-wave infrared (SWIR), and/or another imaging device. In some embodiments, additional images and/or image streams may be obtained during at least a portion of the time period t1 to t2. In some embodiments, the additional images may comprise red-green-blue (RGB) images, monochrome images, ultraviolet (UV) images, multi-spectral images, and/or hyperspectral images.

In some embodiments, image data are processed to extract one or more values associated with at least one of the pixels in each image. In some embodiments, at least one of the values may be extracted at a point in time and/or as a time series over part of all of the period t0 to t2.

In some embodiments, the one or more values may be translated into one or more feature vectors, including one or more time-dependent feature vectors.

In some embodiments, the plurality of feature vectors may be compared with predetermined features or feature vectors associated with a plant tissue state e.g., healthy state. In some embodiments, a state of one or more regions of the plant tissue may be determined, based, at least in part, on the comparison.

In some embodiments, the one or more feature vectors for each pixel are clustered into one or more clusters indicating the clusters as regions of plant tissue states (e.g. healthy vs. unhealthy) of the imaged plant tissue. In some embodiments, differentiation between healthy and unhealthy states of the imaged plant tissue are based on values obtained from a single pixel.

In some embodiments, the present disclosure provides for an output which indicates the states of one or more regions of the a thermal-imaged plant tissue. In some embodiments, the output may comprise an image comprising a graphical representation of the one or more regions, based, at least in part, on the identified plant tissue state associated with each region. For example, in some embodiments, the boundaries of each region may be demarcated, and/or part or the whole of the region may be presented using one or more color schemes. In some embodiments, one or more of the regions may comprise an area of plant tissue corresponding to a single imaged pixel. In some embodiments, the graphical representation may be generated as a thermal image, an RGB image, and/or another and/or a different type of image. In some embodiments, the boundaries of the identified plant tissue states are mapped on a plant tissue state distribution map. In some embodiments, the boundaries of the identified plant tissue states are mapped in the form of a graph, such as, for example, a histogram.

In some embodiments, a machine learning classifier may be trained on a dataset comprising feature vector sets associated with a plurality of plant tissues, wherein the training dataset may be labelled with one or more plant tissue states present in the several regions of the plant tissues. In some embodiments, a trained classifier of the present disclosure may then be applied to a target feature set from a target agricultural product, to determine the presence of the one or more physiological or pathological parameters in the target plant tissue.

In some embodiments, the actively changing the temperature of the plant tissue comprises actively heating or actively cooling the portion of plant tissue during at least a portion of the imaging period of time.

In some embodiments, the processing is performed for each pixel of the obtained image. In some embodiments, the processing includes extracting a plurality of pixel-level values for each pixel, that represent a quantification of a physiological or pathological parameter.

In some embodiments, a method for real-time sorting of plants comprises receiving a sequence of thermal data of a plant, wherein the sequence is sampled at least one location of the tissue while the tissue is being thermally disturbed, processing the thermal data to derive thermal values associated with each of the tissue locations, deriving at least one thermal variable at least one location on the plant, based, at least in part, on the processing, calculating a variance value of all the thermal variables associated with each of the locations and determining a state of the plant based on at least one location at which the variance value exceeds a predetermined threshold.

In some embodiments, the method includes calculating a set of thermal features of each of the tissue locations based, at least in part, on the at least one thermal variable. In some embodiments, the method includes calculating a variance value of all the sets of thermal features associated with each of the locations and determining a state of the plant based on at least one location at which the variance value exceeds a predetermined threshold.

In some embodiments, a method for processing consecutive frames of obtained thermal images for real time sorting of plants based on plant tissue states comprises acquiring a sequence of thermal images over a period of time of at least a portion of the plant tissue. In some embodiments, extracting pixel values from the thermal images, for each pixel and/or point of measurement, generating feature vectors representing plant tissue cell thermal properties over the period of time, clustering pixels having similar features and reflecting the pixel clusters onto corresponding regions in the imaged agricultural product. In some embodiments, the method comprises determining a plant tissue state of at least one of the regions based on comparing the features to known feature sets of plant tissue states. In some embodiments, the method comprises generating an output e.g., a graphic representation of the plant tissue state in one or more of the regions. In some embodiments, the method comprises using trained machine learning classifiers to classify a state of plant tissue in each region.

In some embodiments, the method includes generating a map representing distribution of the physiological or pathological parameter over the portion of the plant tissue within the imaged field of view (FOV). In some embodiments, the method includes processing the distribution over the map of pixel-level values and identifying clusters of values each cluster being within a same pixel value range and associated the identified values with a specific plant tissue type. In some embodiments, clusters of pixels in the thermal image sharing a same numerical value are associated with corresponding clusters of specific plant tissue cell types.

In some embodiments, the distribution processing of the pixel-level values is based on calculation of variance between the calculated pixel-level values.

In some embodiments, the generated an output e.g., a graphic representation of the plant tissue state in one or more of the regions, provide information regarding one or more physiological parameters associated with the integrity or the state of health of the agricultural product. In some embodiments, the thermal image frames are obtained during movement of the agricultural product. In some embodiments, the thermal image frames are obtained without stopping movement of the agricultural product.

According to some embodiments of the invention, the method comprises changing the temperature of an plant over a period of time. In some embodiments, the method comprises actively changing the temperature of an agricultural product. In some embodiments, the method comprises changing the temperature of an plant prior to the imaging period of time. In some embodiments, the actively changing of the temperature of the plant comprises actively heating or actively cooling the plant during at least a portion of the imaging period of time. In some embodiments, the actively changing of the temperature of the plant comprises actively heating or actively cooling the plant before and/or during at least a portion of the imaging period of time. In some embodiments, the actively changing of the temperature of the plant comprises actively cooling followed by actively heating the plant before and/or during at least a portion of the imaging period of time. In some embodiments, the actively changing of the temperature of the plant comprises actively heating followed by actively cooling the plant before and/or during at least a portion of the imaging period of time. In some embodiments, the actively changing the temperature of plant comprises actively heating during at least a portion of the imaging period of time (e.g., t0-t1) followed by actively cooling the plant during at least a portion of the imaging period of time (e.g., t1-t2). In some embodiments, the imaging period of time (e.g., t1-t2) of actively cooling immediately follows the imaging period of time (e.g., t0-t1) of actively heating.

According to some embodiments of the invention, the one or more times or periods of time (durations) at or during which the thermal image frames are obtained comprise a pre-active temperature-changing period of time, a period of time during the active temperature-changing and a post temperature-changing period of time. In some embodiments, one or more thermal image frames are obtained at a time at which the plant temperature peaks. In some embodiments, one or more thermal image frames are obtained at a time at which the plant exhibits a rapid change over time in the generated output indicative of a point of Rapid Image Change (MC).

In some embodiments, the thermal image frames are super imposed on one or more digital white image frames obtained concurrently with the thermal image frames.

According to an aspect of some embodiments of the present invention, the method comprises obtaining a 3D image of plants within an FOV of a 3D camera and processing 3D digital data received from said 3D images. In some embodiments, the obtained 3D digital data comprises information regarding number, size and location of one or more plant within the FOV of the thermal imager of imaged plants.

According to an aspect of some embodiments of the present invention there is provided a method for differentiating between healthy and unhealthy (unhealthy and/or bruised) plants. In some embodiments, the method includes obtaining a thermal image of plants within a field of view (FOV) of a heat pattern emitted from an agricultural product. In some embodiments, the method comprises actively changing a temperature of at least a portion of the plant over a set period of time. In some embodiments, the method includes actively heating or actively cooling at least a portion of agricultural product. In some embodiments, the method includes obtaining the thermal images (frames) over a set period of time. In some embodiments, the method includes processing consecutive frames of the thermal image and extracting information regarding one or more changes in the thermal patterns within a set period of time.

In some embodiments, the processing is performed on each plant within the obtained image. In some embodiments, the method includes generating a graph for each plant representing the change in thermal image of the plant over the set period of time based on generated feature vectors representing plant tissue cell thermal properties over the period of time. In some embodiments, the method includes performing a comparative processing for curves from one or more plants and identifying groups of plants having similar curve patterns associated with the state of health of agricultural product.

In some embodiments, the method comprises actively heating the plant and allowing the plant to cool passively. In some embodiments, processing (as explained in greater detail elsewhere herein) of imaging frames obtained during the period of active heating and passive cooling is expressed by a graph curve having a growth portion, a peak and a decay portion. In some embodiments, a thermal imaging system comprises a processor and a computer program product configured to execute the comparative processing on the growth portion only of the resulting curve. In some embodiments, the comparative processing is executed only on the decay portion of the resulting curve. In some embodiments, the computer program product of the processor is configured to execute the comparative processing on the curve peak temperature only at the meeting point of the growth portion and the decay portion of the resulting curve.

Alternatively, and optionally in some embodiments, the method comprises actively cooling the plant and allowing the plant to warm up passively. In some embodiments, processing of imaging frames obtained during the period of active cooling and passive warming is expressed by a graph curve having a decay portion, a trough (minimum point) and a growth portion. In some embodiments, a thermal imaging system comprises a processor and a computer program product configured to execute the comparative processing on the growth portion only of the resulting curve. In some embodiments, the comparative processing is executed only on the decay portion of the resulting curve. In some embodiments, the computer program product of the processor is configured to execute the comparative processing on the curve trough (minimum temperature) only at the meeting point of the growth portion and the decay portion of the resulting curve.

In some embodiments, the method includes incrementally actively heating or cooling the portion of agricultural product. In some embodiments, the method includes obtaining thermal images (frames) over a set period of time. In some embodiments, the method includes processing consecutive frames of the image obtained during each actively heating or cooling increment and extracting information regarding a change within the heating increment in one or more physiological parameters associated with the state of health of the agricultural product.

According to an aspect of some embodiments of the present invention there is provided a method for differentiating between healthy and unhealthy plants. In some embodiments, the method includes actively heating the agricultural product. In some embodiments, heating the plants includes applying heating energy (e.g., Infrared light) to the agricultural product.

In some embodiments, the method includes processing consecutive frames of the image obtained over the period of time and extracting information regarding a rate of thermal diffusion the heated plant during the set period of time. In some embodiments, processing consecutive frames of the image obtained over the set period of time includes associating the information regarding the rate of thermal diffusion in the plant with one or more parameters associated with the state of health of the agricultural product. In some embodiments, the method includes identifying groups of pixels sharing a diffusion rate within a given range that is associated with the state of health of agricultural product.

According to an aspect of some embodiments of the present invention there is provided a method for differentiating between healthy and unhealthy plants. In some embodiments, the method includes heating the plant for a first predetermined period of time (e.g., t0-t1). In some embodiments, the heating is immediately followed by actively cooling the plant for a second predetermined period of time (e.g., t1-t2). In some embodiments, cooling the plant includes applying cooling energy (e.g., sprays or contact coolants) to the agricultural product. In some embodiment, the method includes obtaining the thermal image of at least a portion of the plant within the FOV over a set period of time.

In some embodiments, the method includes processing consecutive frames of thermal images (frames) obtained over the period of time and extracting information regarding a rate of thermal diffusion over the surface and/or within the plant during the predetermined period of time. In some embodiments, processing consecutive frames of the image obtained over the predetermined period of time includes associating the information regarding the rate of thermal diffusion in the plant with diseases of the agricultural product. In some embodiments, the method includes identifying groups of plants sharing a diffusion rate within a given range that is associated with the state of health of the agricultural product.

According to an aspect of some embodiments of the present invention there is provided a method for differentiating between healthy and unhealthy plants. In some embodiments, the method includes heating the agricultural product. In some embodiments, heating the plant includes applying heating energy (e.g., Infrared light) to a predetermined depth within the agricultural product. In some embodiment, the method includes obtaining the thermal image of the portion of plant at various depths between the plant surface and the predetermined depth over a set period of time.

Alternatively, and optionally, in some embodiments the method for differentiating between healthy and unhealthy plant includes following the heating of the plant by actively cooling the agricultural product. In some embodiments, cooling the plant includes applying cooling energy (e.g., sprays or contact coolant) to a predetermined depth within the agricultural product. In some embodiment, the method includes obtaining thermal images (frames) of the portion of plant at various depths between the plant surface and the predetermined depth over a set period of time.

In some embodiments, the method includes processing consecutive frames of the thermal image obtained at any specific depth over the period of time and extracting information regarding a rate of thermal diffusion throughout a layer of plant at the specific depth during the set period of time. In some embodiments, processing consecutive frames of the image obtained over the set period of time includes associating the information regarding the rate of thermal diffusion within the plant with associated with the state of health of the agricultural product. In some embodiments, the method includes identifying groups of voxels sharing a diffusion rate within a given range that is associated with the state of health of the agricultural product.

According to an aspect of some embodiments of the present invention there is provided a computer program product comprising a non-transitory computer-readable storage medium having program code embodied therewith, the program code executable by at least one hardware processor to analyze a heat pattern of one or more thermal images (frames) of at least a portion of plant within a field of view (FOV).

In some embodiments, the computer program product is executable to calculate a thermal parameter (e.g., temperature) from information received from each pixel of an obtained image. In some embodiments, the program product is executable to use the calculated thermal parameter to further calculate a numerical value associated with a physiological parameter of the agricultural product. In some embodiments, the program product is executable to generate a map based on the calculated numerical values associated with the physiological parameter of the agricultural product. In some embodiments, the program product is executable to indicate groups of pixels having numerical values associated with the parameters within a given range of parameters associated with the state of health of the agricultural product.

According to an aspect of some embodiments of the present invention there is provided a computer program product comprising a non-transitory computer-readable storage medium having program code embodied therewith, the computer program product executable by at least one hardware processor to analyze a heat pattern of a thermal image of a portion of plant within a field of view (FOV).

In some embodiments, the computer program product is executable to analyze the heat pattern register and calculate a change in a thermal parameter (e.g., temperature) received from at least one pixel over a set period of time. In some embodiments, the program product is executable to calculate the change in the thermal parameter from a plurality of the image pixel frames taken over the set period of time. In some embodiments, the program product is executable to use the identified thermal parameter to calculate a numerical value associated with the state of health of the agricultural product.

In some embodiments, the program product is executable to generate a curve based on the calculated numerical values associated with the change in the physiological parameter of the plant over the set period of time. In some embodiments, the program product is executable to indicate groups of pixels having similar curves indicating the change in the numerical values associated with the parameters within a given range associated with a specific plant type of disease.

### The System

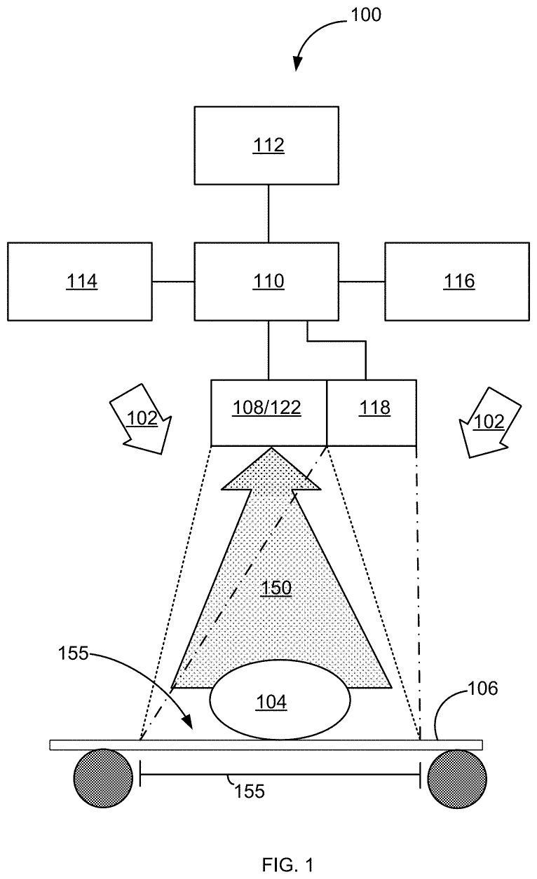

Reference is now made to FIG. 1. Which is a simplified diagram of a thermal imaging system for real-time plants. In some embodiments, thermal imaging system 100 for real-time plants comprises a thermal imager 108 that images one or more actively heated or cooled plants 104 on a moving sorting path 106. In the exemplary embodiment depicted in FIG. 1, sorting path 106 comprises a conveyor belt however in some embodiments, sorting path 106 comprises sorting tables, sorting machines or any other sorting configuration. In some embodiments, the thermal imager 108 images one or more plants 104 on a moving sorting path without stopping (e.g., without stopping a sorting line or conveyor belt). In some embodiments, thermal imager 108 is in communication with a processor 110. Thermal imaging system 100 processor 110 is configured to process and analyze thermal images obtained by thermal imager 108 and generate an output, for example, on a display 112.

As illustrated in FIG. 1, a thermal imaging system 100 for real-time plants comprises a heating/cooling source 102 directed at an plant 104 to be analyzed.

In some embodiments, active heating can include one or more heating methods selected from a group of heating methods including radiation, convection and conduction. Heat source 102 can be for example, any suitable heat source such as, for example, High Radiant Flux Density 400 nm Violet LED Emitter LZP-DOUB00-00U5 manufactured by LED Engin®, Inc., San Jose Calif. 95134, USA or any InfraRed (IR), Radio Frequency (RF), Ultrasound (US), Fluid flow over the agricultural product, heating pipes or other carriers, etc.

In some embodiments, active cooling can be applied for example, by evaporation (e.g., alcohol sprays), local coolant sprays (nitrogen), cooling fluid flow over the agricultural product, cooling pipes or other carriers, refrigeration, etc.

In some embodiments and as shown in FIG. 1, system 100 digital thermal imager 108 images thermal radiation 150 emitted from the plant 104. In some embodiments, imager 108 is a video thermal imager configured to generate consecutive frames of thermal images obtained of the plant 104 within a field of view (FOV) 155 of thermal imager 108 over a set period of time. In some embodiments, thermal imager 108 comprises a digital microscope thermal imager 108. In some embodiments, thermal imager 108 can be any suitable digital imager such as, for example, a PI 450 Thermal Infrared Video Camera by Optris®, Portsmouth, N.H. 03801 USA. In some embodiments, system 100 comprises a visible light camera. In some embodiments, digital imager 108 comprises any suitable thermal sensor for example, MM, Ultrasound, Thermocouple or any other sensor that measures temperature.

In some embodiments and as shown in FIG. 1, system 100 comprises a 3D imager 118 configured to image plants within FOV 155 of imager 108 and communicate the 3D image data to processor 110. In some embodiments, the obtained 3D digital data comprises information regarding number, size and location of the plant within FOV 155 of thermal imager 108. As explained in greater detail herein, processor 110 is configured to factor in the data obtained from 3D imager 118 (e.g., change in location within the FOV of the plant or relative sizes of plants within the FOV) when processing the thermal image data of imaged plants obtained from thermal imager 118, e.g., to normalize thermal image data obtained from thermal imager 118 of imaged plants.

In some embodiments and as explained in greater detail elsewhere herein, system 100 comprises a source of illumination 114 that demarcates the plant identified by processor 110 as unhealthy (unhealthy and/or blemished). In some embodiments and as explained in greater detail elsewhere herein, system 100 comprises a source of ablative energy 116 to physically mark unhealthy (unhealthy and/or blemished) agricultural product. In some embodiments, source of illumination 114 and source of ablative energy 116 are generated from a single source (e.g., laser).

In some embodiments, imager 108 is calibrated by imaging a surface 155 of sorting path 106. In some embodiments, surface 155 is imaged bare. In some embodiments, surface 155 is made of a material with known thermal characteristics, in which case imager 108 is calibrated online or offline according to a lookup table.

In some embodiments, and as depicted in FIG. 2, which is a simplified diagram of a thermal imaging system for real-time plants, system 100 includes one or more imaging stages. In some embodiments, the one or more imaging stages occur at different locations along the sorting path in real-time. In some embodiments, the one or more imaging stages occur at one location along the sorting path. A pre-active temperature-changing time (e.g., pre-heating) stage I, an active temperature-changing (e.g., active heating) stage II and a post temperature-changing (e.g., heating) time stage III. In the embodiment depicted In FIG. 2, plant 104 is heated at stage II and allowed to passively cool down at stage III.

In some embodiments, one or more plants pass through stages 1, 2 and 3 while being carried by sorting path 106 in a direction indicated by arrow 250.

In some embodiments, one or more stages I, II and/or III include one or more 3D imagers 118. Processor 110 is configured to obtain and analyze 3D image data from 3D imagers 118 at stages I, II and/or III and factor in the data obtained from 3D imager 118 when processing the thermal image data of imaged plants obtained from thermal imager 118, e.g., data pertaining to movement (rolling) and change of location of an plant on sorting path 106 during transition between stages.

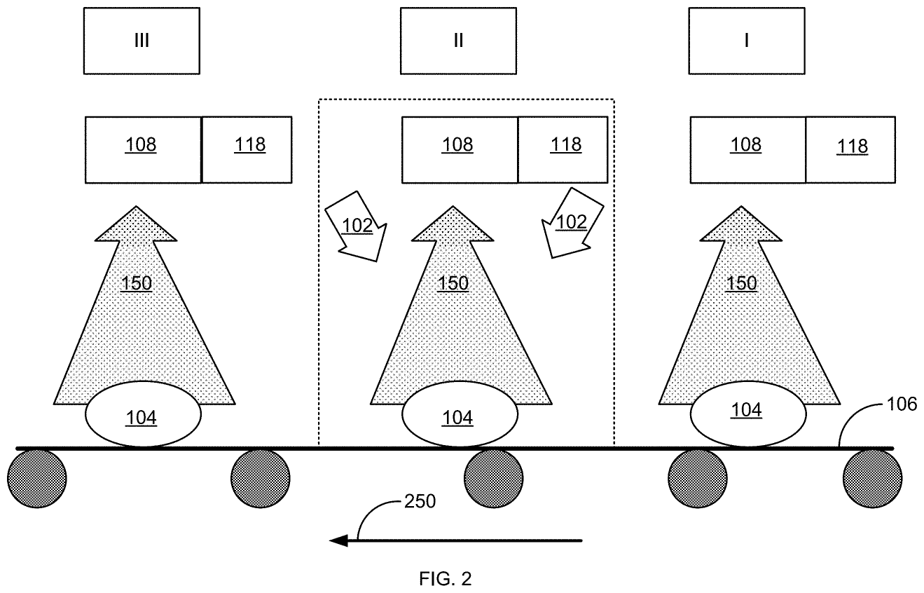

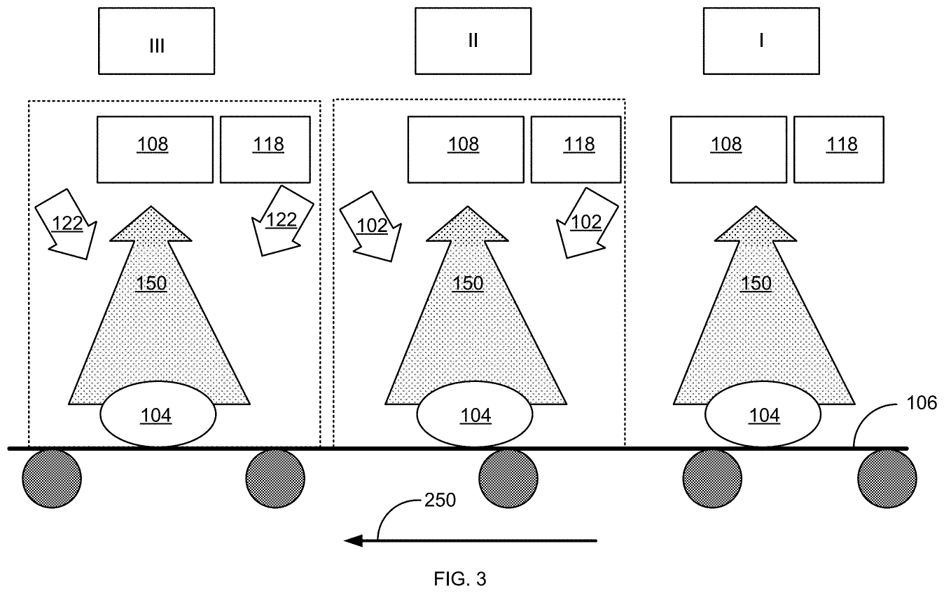

In some embodiments, and as shown in FIG. 3, which is a simplified diagram of a thermal imaging system for real-time plants, system 100 includes one or more imaging stages. A pre-active temperature-changing time (e.g., pre-heating) stage I, an active temperature-changing (e.g., active heating) stage II and an active temperature-changing (e.g., active cooling) time stage III. In some embodiments, stage III begins immediately or soon after active heating is stopped and can provide, for example, post-heating active or passive cooling. In some embodiment, stage III begins immediately after arriving at the point of maximal temperature (peak point). In the embodiment depicted In FIG. 3, plant 104 is heated at stage II and actively cooled at stage III.

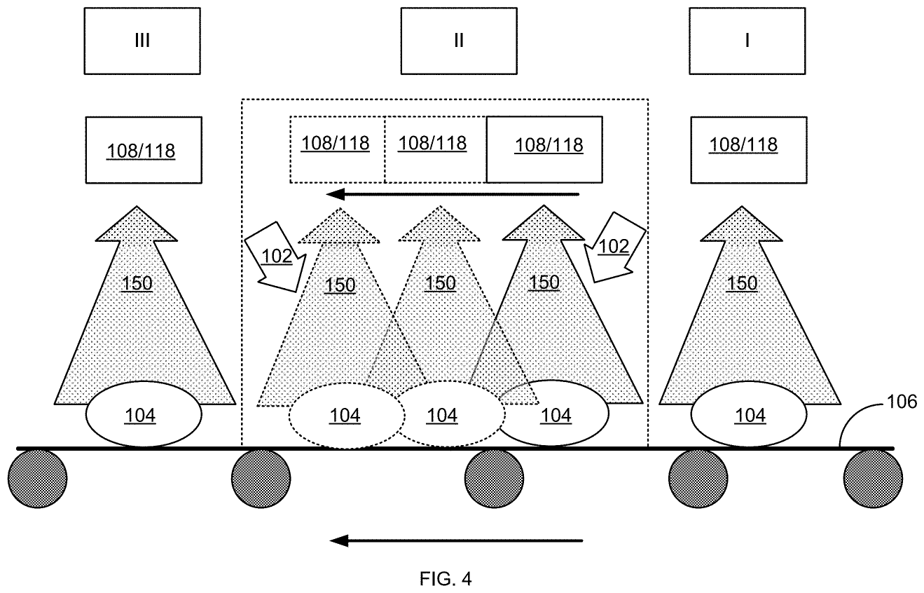

Referring now to FIG. 4, which is a simplified diagram of a thermal imaging system for real-time plants, at one or more stages, system 100 thermal imager 108 and/or 3D imager 118 are configured to move concurrently with and at a speed correspondent with the speed of sorting path 106. In the exemplary embodiment depicted in FIG. 4, imagers 108/118 at stage II move concurrently with sorting path 106 while obtaining one or more images or continuously imaging plants 104. As will be explained in greater detail herein, this allows processor 110 to identify a Temperature Effecting Point (TEP) in time at which blemishes, and superficial imperfections appear in the thermal image.

Processing of a Thermal Image (Frame) Obtained from a Single Pixel

For simplicity of explanation the examples hereinbelow refer to an IR digital imager. However, as explained elsewhere herein, any other suitable thermal imager can be used.

In some embodiments, digital thermal imager 108 comprises one or more pixel arrays. The pixel arrays react to IR radiation emitted from the imaged plant 104. One or more pixels react to IR radiation emitted from a corresponding segment (Sₚ) of the imaged plant 104 within an FOVp of the pixel. In some embodiments, heat source 102 is configured to gradually actively heat plant 104 over a set period of time after which active heating is stopped and the plant is allowed to cool passively during which time the plant temperature returns to the temperature prior to initiation of the active heating. Throughout the heating and cooling period of time, thermal imager 108 obtains a series of consecutive frames of a thermal image of plant 104.

In some embodiments, each consecutive thermal frame in the obtained series of thermal images is time-stamped and therefore a series of two or more frames obtained over a period of time provides information regarding changes in recorded thermal parameters of the agricultural product. In some embodiments, system 100 processor 110 is configured to analyze the recorded thermal parameters and map a thermal change behavior of the agricultural product. In some embodiments and as explained in greater detail elsewhere herein, processor 110 is configured to define a plant disease type based on the thermal behavior map of the imaged agricultural product.

In some embodiments, a cooling source 122 is configured to actively cool plant 104 over a set period of time after which active cooling is stopped and the plant is allowed to passively warm up during which time the plant temperature returns to the temperature prior to initiation of the active cooling. Throughout the cooling and warming up period of time, thermal imager 108 obtains a series of consecutive frames of a thermal image of plant 104.

Referring back to the exemplary embodiment depicted in FIG. 2, plant 104 is actively heated at stage II and allowed to passively cool down at stage III. Alternatively, and optionally, and as shown in the exemplary embodiment depicted in FIG. 3, plant 104 is actively heated at stage II and actively cooled at stage III.

In some embodiments, each consecutive thermal frame in the obtained series of thermal images is time-stamped and therefore a series of two or more frames obtained over a period of time provides information regarding changes in recorded thermal parameters of the agricultural product. In some embodiments, system 100 comprises a processor 110 configured to analyze the recorded thermal parameters and map a thermal behavior of the agricultural product. In some embodiments and as explained in greater detail elsewhere herein, processor 110 is configured to define a plant type of the imaged plant based on the thermal behavior map of the imaged agricultural product.

In some embodiments, Thermal imaging system 100 processor 110 comprises a non-transitory computer-readable storage medium having program product embodied therewith. The program product is executable by thermal imaging system 100 processor 110 to analyze e.g., compare and map differences for each pixel between the consecutive time-stamped frames of the thermal image of the portion of plant.

Thermal imaging system 100 processor 110 is configured to process electronic signals received from each pixel for each consecutive frame of FOVp in accordance with the time stamp of each frame and generate a graph indicating a change in the IR radiation emitted from each segment Sₚ of plant 104 over the set period of time.

In some embodiments, thermal imaging system 100 processor 110 uses one or more algorithms that use various mathematical expressions to approximate the obtained results to the signals received from the imager pixels and generate accurate mapping of the imaged plant type. Such mathematical expressions include, for example, the following mathematical expression, which is based on Pennes' equation of bio-heat transfer:

T(t)=a+be^{ct}+dt

Wherein (dt) may be normalized by time and variables (a), (b), (c) and (d) are variables derived from Pennes' bioheat transfer equation which is a widely accepted temperature profiling equation for biological plants. Variables (a), (b), (c) and (d) are used herein for the purpose of clarity and simplicity, by way of example but not by way of limitation, and can include any number or combination of variables and be any type. For example, variables (a), (b), (c) and (d) can be at least any one of external parameters affecting thermal behavior of the plant tissue e.g., environmental temperature, external heat source and interior and the time-dependent thermal gradient between environment and object and/or intrinsic plant parameters affecting thermal behavior of the plant tissue (thermal parameters) e.g., plant density, heat capacity, thermal conductivity factor, heat transfer coefficient and the heat transfer surface area (m2).

In some embodiments, a plurality of features may be calculated based, at least in part, on the variables (e.g., variables a, b, c and d), including, but not limited to, features representing various derivative values of the variables, features representing noise in the variables, features based on decay equations, features based on Fourier series, as well as correlative features based on the variance of the features.

The following is an example for such a derivation based on Pennes' equation expressed as:

$$ρ {c}_{ρ} \frac{\partial T}{\partial t} = \frac{\partial K}{\partial x} \frac{\partial T}{\partial x} + k \frac{{\partial }^{2} T}{\partial {x}^{2}} + \frac{\partial k}{\partial y} \frac{\partial T}{\partial y} + k \frac{{\partial }^{2} T}{\partial {y}^{2}} + \frac{\partial k}{\partial z} \frac{\partial T}{\partial z} + k \frac{{\partial }^{2} T}{\partial {z}^{2}} - ρ c ω ( T - {T}_{a} ) + g ( x , t )$$

In some embodiments, the following assumptions are taken:

a) That the lateral contributions and/or the heat generation are negligible (metabolism time scale is one minute [1]) and therefore obtain the following expression:

$$ρ {c}_{ρ} \frac{\partial T}{\partial t} \approx \frac{\partial k}{\partial z} \frac{\partial T}{\partial z} + k \frac{{\partial }^{2} T}{\partial {z}^{2}} - ρ c ω ( T - {T}_{a} ) \\ b ) that$$

$$\frac{\partial k}{\partial z} \frac{\partial T}{\partial z} + k \frac{{\partial }^{2} T}{\partial {z}^{2}} \approx hC ( T - {T}_{env} ) - k \frac{T ( z ) - T ( z - Δ z )}{{( Δ z )}^{2}} \approx hC ( T - {T}_{env} ) - kf ( t ) ( T - {T}_{c} )$$

where C is area and (h) is the heat transfer coefficient, Tc is the core temperature, and

c) We assume that f(t) changes slowly with time.

Under the disclosed assumptions:

$$ρ {c}_{ρ} \frac{\partial T}{\partial t} \approx hC ( T - {T}_{env} ) - kf ( t ) ( T - {T}_{c} ) - ρ c ω ( T - {T}_{a} ) , \\ \frac{\partial T}{\partial t} \approx \frac{hC - kf ( t ) - ρ c ω}{ρ {c}_{ρ}} T + \frac{- {hCT}_{env} + kf ( t ) {T}_{c} + ρ c ω {T}_{a}}{ρ {c}_{ρ}} , \\ \frac{\partial T}{\partial t} \approx - cT + Ac , \\ Where$$

$$c \approx - \frac{hC - kf ( t ) - ρ c ω}{ρ {c}_{ρ}} , A \approx \frac{- {hCT}_{env} + kf ( t ) {T}_{c} + ρ c ω {T}_{a}}{hC - kf ( t ) - ρ c ω} , B \approx {T}_{i} - A$$

Equation (4.65) in Analytical Bioheat Transfer: Solution Development of the Pennes' Model, Sid M. Becker, Chapter 4 agrees with this formulation in the limit 4αt=12; 1->0.

T (t˜=0) may also be approximated with a linear function or a higher order polynomial:

T(t≅0)≈B+A−cBt=Tᵢ−cBt

We express T (t) as an exponent for short times (e.g., t may be between 0 and 40 seconds, 10 and 30 seconds, 15 and 25 seconds or any number of seconds in between).

In another example and in some embodiments, variable (a) may express initial conditions at the point of transfer from active heating/cooling to passive cooling down or warming of imaged plant and is not time dependent. In some embodiments, variables (b) and/or (c) express a combination of plant physiological parameters such as, for example, density (p), specific heat (C) and thermal conductivity (K).

As explained in greater detail elsewhere herein, in some embodiments, extracted variables (a), (b), (c), (d) and other contributing variables, groups of the same variable or groups of variables from one or more thermal images together with one or a combination of mathematical expressions are analyzed by a computer program product of processor 110, using data mining processes, e.g., to cross-reference data, perform data cleansing, and generate an output in a form of a map indicating and/or identifying various plant disease types within the imaged plant area.

In some embodiments, the following expressions are used for plants without an internal heat source (e.g., fully ripe):

$$\begin{matrix} T ( t ) = a + b {e}^{- c t} & 1. \\ \frac{T ( t ) - Ti}{T ∞ - Ti} = 1 - {e}^{- c t} [ 1 - \frac{2}{π} * ( \sqrt{c} * \sqrt{t} - \frac{1}{3} * {c}^{\frac{3}{2}} * {t}^{\frac{3}{2}} + \frac{1}{10} * {c}^{\frac{5}{2}} * {t}^{\frac{5}{2}} ) ] & 2.\end{matrix}$$

### Wherein:

Ti is initial temperature of the body.

T∞ is ambient temperature.

$$Parameter C = \frac{{h}^{2} \cdot α}{{k}^{2}} [ \frac{1}{s} ]$$

Where

$$h [ \frac{w}{{m}^{2} \cdot k} ]$$

is convective heat transfer.

$$k [ \frac{w}{m \cdot k} ]$$

is thermal conductivity.
3.

$$θ ( t ) = \frac{T ( t ) - {T}_{ambient}}{{T}_{i} - {T}_{ambient}} = b * {e}^{- c t} ( With normalize tempuratures ) .$$

Commonly, plants are collected prior to ripening and continue the ripening physiological process en route from the field to the consumer. The ripening process generates heat. In some embodiments, the following expressions are used for plants with an internal heat source:

T(t)=a+be^{-ct}−dt 1.

T(t)=a+be−^{-ct}−de^{-ht}^{²} 2.

T(t)=a+be^{-ct}*de^{-ht}^{²} 3.

T(t)=a+be^{-ct}−de^{-gt}·e^{-ht}^{²} 4.

Wherein (h) is a convection factor (e.g., transfer of heat from plant tissue to air) and is therefore dependent on ambient temperature.

In some embodiments, during the processing, a computer program product of processor 110 compares a generated output map based on each obtained image to a gold standard and elects to adjust the processing process (e.g., by changing selected variables, selected mathematical calculation combinations), generate or not to generate the output map.

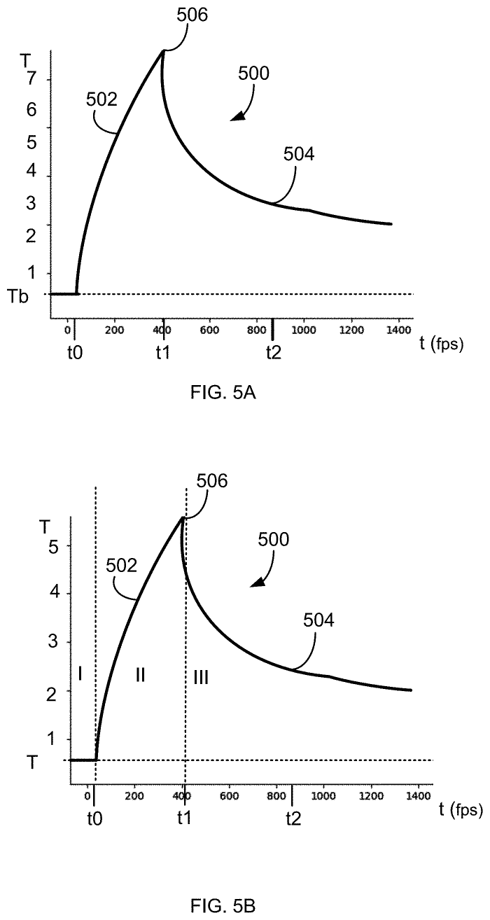

Graph curves in FIGS. 5A, 5B, 6, 7, 8A, 8B, 10A-10C, 12B, 15A, 15B and 16 represent temperature (T⁰c) changes over time (t) indicated by Image Frames per Second (FPS). For example, in cases in which images are obtained at a rate of 25 FPS, every 25 frames represent one second.

Reference is now made to FIGS. 5A and 5B, which are graphs of thermal curves associated with biothermal behavior of a heated plant in accordance with some embodiments of the invention. In the exemplary embodiments depicted in FIGS. 5A and 5B, curves 500 exhibit a thermal curve indicating thermal behavior of a healthy tuber, in this case—a potato during active heating (growth portion 502), arriving at a temperature peak 506 and allowing to passively cool (decay portion 504).

Though the example described herein relates to potatoes, in some embodiments, the method includes real-time sorting of plants of a single kind or type (e.g. potatoes or apples or oranges). In some embodiments, the method includes real-time sorting of plants of the same kind or type and different varieties (potatoes and yams, Granny Smith apples and Red Delicious apples). In some embodiments, the method includes real-time sorting of plants of a mix of kinds or types (e.g., apples and potatoes, oranges and pears).

In some embodiments, curve 500 exhibits a change in temperature (T) of a plant from a base temperature (Tb) within a segment of plant (Sp) based on IR radiation emitted from each imaged Sp of plant 104 over the set period of time (t0 to t1). In some embodiments, curve 500 expresses biothermal behavior of plant in response to heating over a set period of time (t0 to t1) and comprises a growth portion 502 in response to heating, a decay portion 504 during a cooling period of time (t1 to t2) and a peak temperature 506 at the meeting point (t1) of growth portion 502 and decay portion 504.

As is explained in greater detail elsewhere herein, the set period of time (t₀ to tₙ) need not necessary reflect a period of heating followed by a period of cooling and may be broken down into periods of time comprising various modalities of temperature change.

As explained in International Patent Application No. PCT/IL2015/050392 to the same inventors, various types of plants exhibit specific biothermal behavior expressed by one or more of a specific growth portion 502, a specific decay portion 504 and a specific curve peak temperature 506. In some embodiments, the thermal imaging system 100 images the plant over a total period of time (t0 to t2) and processes the electronic signals received from the pixel for each consecutive frame of FOVp in accordance with the time stamp of each frame and generates a growth portion 502 specific for the imaged agricultural product. Similarly, in some embodiments, thermal imaging system 100 processes the electronic signals received from the pixel for each consecutive frame of FOVp in accordance with the time stamp of each frame and generates a decay portion 504 specific for the imaged agricultural product. Accordingly, thermal imaging system 100 can combine specific growth portion 502 and decay portion 504, calculate a meeting point of curve portions 502 and 504 and generate numerical values expressing the position of peak temperature 506 on the generated curve 500.

As disclosed elsewhere herein, in some embodiments, the method implemented via system 100 comprises actively changing the temperature of plant during at least a portion of the imaging period of time (e.g., t0 to t1). In some embodiments, obtained frames provide information regarding changes in plant physiological parameters over the imaging period of time. In some embodiments, actively changing the temperature of a plant comprises actively heating or actively cooling the portion of plant during at least a portion of the imaging period of time.

In some embodiments, data can be extracted, as explained elsewhere herein, from both active heating and active cooling sessions thus increasing the accuracy of the maps outputted by computer program product of processor 110.

For simplicity of explanation the examples hereinbelow refer only to method embodiments comprising heating. However, all disclosed method embodiments, can be implemented in the same manner replacing active heating with active cooling. FIG. 5B, demonstrates the stage numbers I, II and III at which a corresponding section of thermal curves associated with biothermal behavior of a heated plant are obtained.

In the exemplary embodiment depicted by curve 500 in FIG. 5B, stage I comprises a pre-active temperature-changing (heating) period of time (steady state) at which no temperature change takes effect, stage II comprises a period of time of active temperature-changing (heating) exhibited by a growing curve portion 502 up to a peak 506 and stage III comprises a post temperature-changing (passive cooling) period of time represented by a decay portion 504 of thermal curve 500. In some embodiments, stage III begins immediately after plant temperature peak 506. In some embodiments, one or more thermal image frames are obtained at a time at which the plant temperature peaks 506. In some embodiments, one or more thermal image frames are obtained at a time period at which the plant exhibits a rapid temperature change (point of Rapid Image Change (MC)).

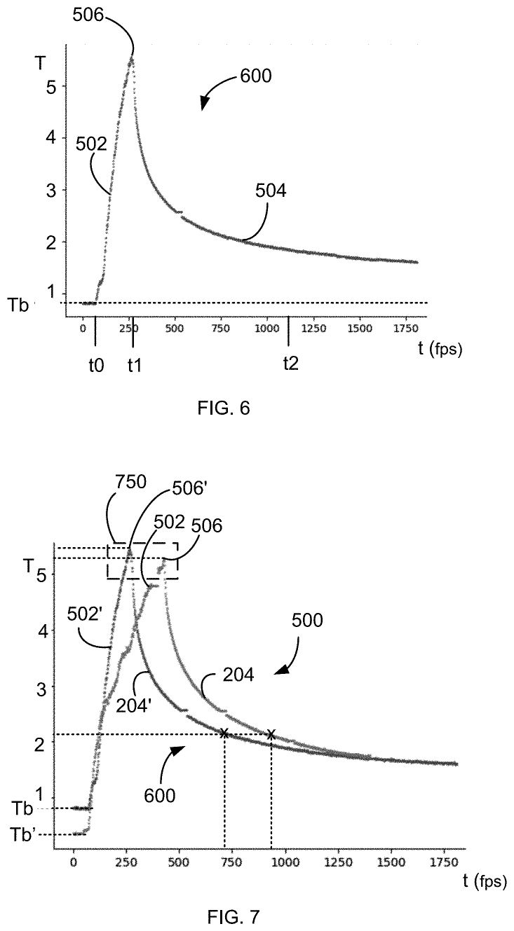

Reference is now made to FIG. 6, which is a graph of a thermal curve associated with biothermal behavior of a heated plant in accordance with some embodiments of the invention. In the exemplary embodiments depicted in FIG. 6, curves 500 exhibit a thermal curve indicating thermal behavior of a healthy potato during active heating (growth portion 502), arriving at a temperature peak 506 and allowing to passively cool (decay portion 504).

In some embodiments, the curve 600 generated by thermal imaging system 100 processor 110 is a thermal signature of a tuber, in this case a potato, infected by Potato Powdery Scab (Spongospora subterranea (Wallr.) Lagerh., f.sp. subterranea Tomlinson). The difference between a healthy potato thermal curve (FIGS. 5A and 5B) and an infected potato (FIG. 6) can be easily appreciated by a comparison between thermal curves 500 and 600 as shown in FIG. 7, which is a graph of thermal curves associated with biothermal behavior of a heated plant in accordance with some embodiments of the invention.

As shown in the exemplary embodiment depicted in FIG. 7, thermal curves of a healthy potato (500) and an infected potato (600) are overlapped and drawn on the same T/t coordinate system to accentuate the differences in the curve patterns. For example, a base temperature of the infected potato (Tb′) is lower than the base temperature of a healthy potato (Tb). However, the peak temperature of the infected potato (506′) is higher than the peak temperature of the healthy potato (506).

It is also noted that the growth portion of the thermal curve of the infected potato (502′) is steeper (i.e., the potato heats up faster) than the growth portion of the thermal curve of the healthy potato (502) arriving earlier at peak temperature 506′.

Differences are also noted by comparing arrival time at one or more temperatures along the growth portion or decay portion of the thermal graphs of each specimen. For example, and as shown in FIG. 7, arrival time at a temperature of approximately 31 degrees Centigrade is marked by an X on the decaying portion of curves 500 and 600. The cooling down of the infected potato is quicker than that of the healthy potato arriving at 31 degrees Centigrade at about 28 seconds (frame 700) from T(0) versus a slower cooling down of the healthy potato arriving at 31 degrees Centigrade at about 36 seconds (frame 900) from T(0). Hence, a thermal signature can be established for a healthy plant and any variance from the established healthy signature can be identified by processor 110 as a “unhealthy” (unhealthy and/or blemished) agricultural product.

Moreover, thermal signatures can be established for a slew of agricultural diseases and a lookup chart compiled so that processor 110 does not only generate a binary output differentiating between a “healthy” or “unhealthy” (unhealthy and/or blemished) plant (e.g., potato), but also identify and point out the type of disease that is ailing the agricultural product.

Accordingly, thermal imaging system 100 can identify thermal behavior curves 500 and, for example 600 as specific to different plant diseases (e.g., Potato Powdery Scab) and can therefore be used to indicate an existence of different plant diseases in the examined agricultural product.

As disclosed elsewhere herein, the variances are exhibited all along the thermal behavior curve and therefore enable processing only portions of the curve such as only growing portion 502, only decay portion 504, only by peak temperature 506 location or any combination thereof.

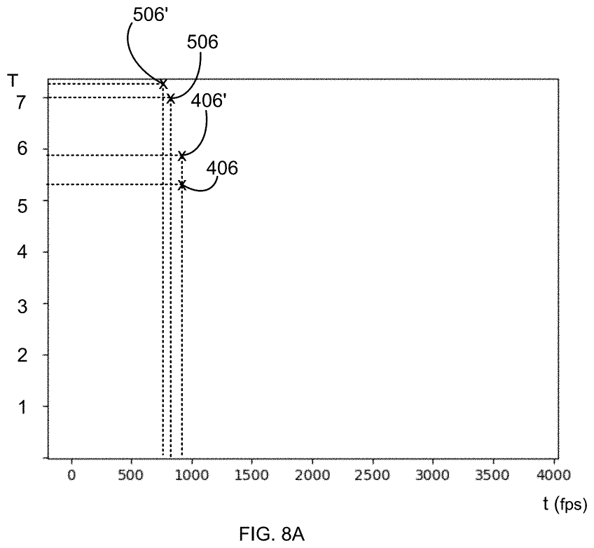

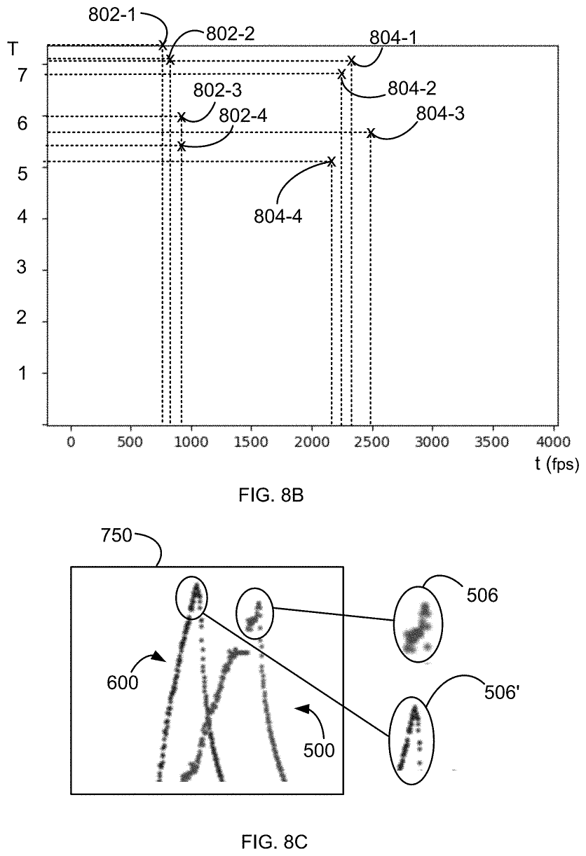

Reference is now made to FIGS. 8A, 8B and 8C, collectively referred to as FIG. 8, which is a graph processing of peak temperature points 506/406 in accordance with some embodiments of the invention compared by being drawn on the same T/t coordinate system. Peak temperature points 506 and 506′ are derived from thermal behavior curves 500/600 respectively and peak temperature points 406 and 406′ are examples of curve peaks described herein for purposes of explanation only. As depicted in the exemplary graph shown in FIG. 8, the variance between peak temperatures 506 and 506′ is expressed in temperature and/or time of arrival at the peak temperature. However, the variance between peak temperatures 406 and 406′ is expressed only in the temperature but peak temperatures 406 and 406′ are shown to have arrived at the same time. Thermal imaging system 100 processor 110 is configured to identify variance in the coordinates of peak temperatures 506/506′ and 406/406′ and thereby be used to indicate an existence of different plant disease types in the examined agricultural product.

In some embodiments and as depicted in FIG. 8C, which is portion 750 (Shown in FIG. 7) of an exemplary embodiment of a pair of thermal behavior curves 500 and 600 obtained from two pixels P1 and P2 and compared by being drawn on the same T/t coordinate system, a computer program product of processor 110 is configured to compare not just the variance between peak temperatures 506 and 506′ expressed in temperature and/or time of arrival at the peak temperature but also analyze the shape of at least a portion of the graph leading to the peak (i.e., of the growing portion) and/or a portion of the graph following the peak (e.g., of the decaying portion).

As explained elsewhere herein, based on the peak shape processing, the computer program product of processor 110 is configured to identify on a generated output map a thermal signature specific to a plant disease type imaged within an FOVp of a pixel, e.g., by identifying a thermal behavior pattern specific to a disease type.

In reference to FIGS. 5A-5B, 6 and 7, in some embodiments, thermal imaging system 100 processor 110 collects data from a plurality of pixels of imager 108 and group the calculated results, e.g., growth portions 502 in response to heating, a decay portions 504 during cooling, peak temperatures 506 at the meeting point of growth portion 502 and decay portion 504 and seasonal noise and define a cutoff lineation between groups displaying close or similar profiles.

In some embodiments, and as shown in FIG. 8B, which is a plurality of peak temperature points (e.g., 506/406) compared by being drawn on the same T/t coordinate system, grouped and identified by thermal imaging system 100 processor 110 as an early peaking group (802-1, 802-2, 802-3 and 802-4) that peaked e.g., under 1000 frames (e.g., at an imaging rate of 25 frames per second 1000 frames are imaged over four seconds) and identified as containing normal plant based on a lookup table generated by thermal imaging system 100 processor 110 as explained elsewhere herein and a late peaking group (804-1, 804-2, 804-3 and 804-4) that peaked e.g., only over 80 seconds and identified based on the lookup table as containing unhealthy agricultural product.

Alternatively, and optionally, in some embodiments, computer program product of processor 110 is configured to compare the variance between peak temperatures 506 and 506′ expressed in temperature and/or time of arrival at the peak temperature to a known reference (e.g., a lookup table).

As depicted in FIGS. 5A-5B, 6 and 7, in some embodiments, peak temperature points such as peak temperature points 506/506′ can also be identified as thermal signatures of specific plant disease types.

In some embodiments and as disclosed elsewhere herein, the graph generated by thermal imaging system 100 processor 110 and shown for example, in FIGS. 5A-5B, 6 and 7 is based, among others Pennes' bioheat equation wherein variables (a), (b), (c) and (d) can be at least any one of the following variables including environmental temperature, external heat source, plant density, heat capacity, thermal conductivity factor, heat transfer coefficient, the heat transfer surface area (m³), the temperature of the object's surface and interior and the time-dependent thermal gradient between environment and object.

### Plant Disease Type Characterization

In some embodiments, a thermal sensor array of pixels of imager 108 images an plant 104. In some embodiments, plant 104 is preheated. Each pixel in the thermal sensor array reacts to the infrared energy focused on it within the pixel FOVp and produces an electronic signal. Thermal imaging system 100 processor 110 receives the signal from each pixel and applies a mathematical calculation to it to create a map of the apparent temperature gradient over the object. In some embodiments, each temperature value is assigned a different color. The resulting matrix of colors is sent to memory of thermal imaging system 100 processor 110 and to a system display as a thermal map (temperature distribution image) of plant 104.

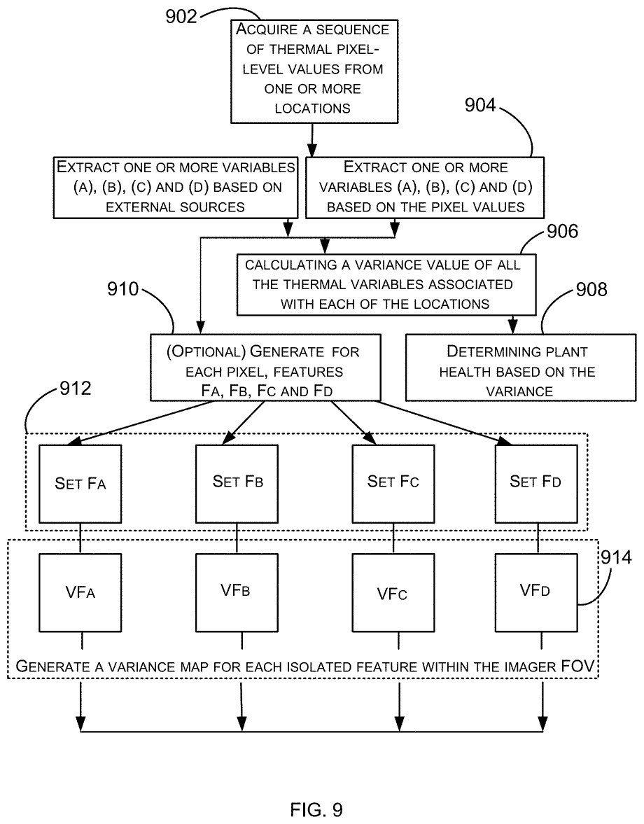

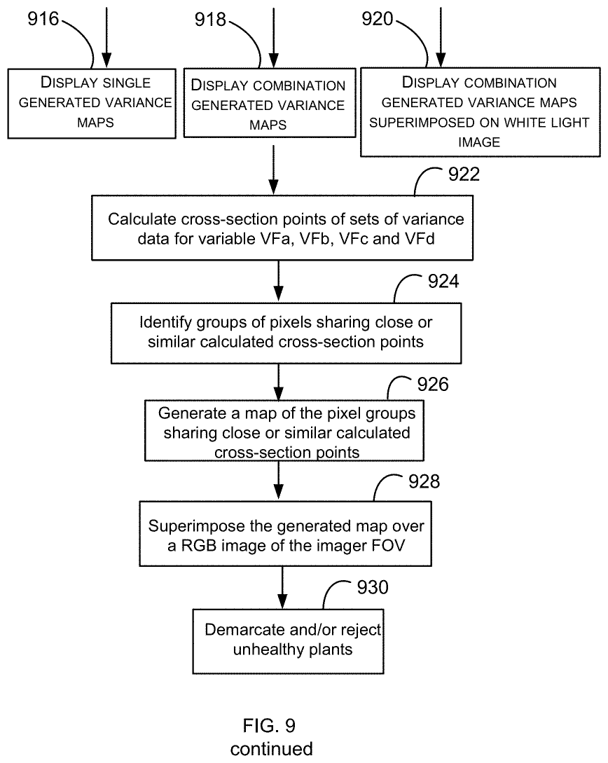

The following is an example of the above described method for differentiating between plant disease types. Reference is now made to FIG. 9, which is an exemplary simplified flow chart illustrating operation of thermal imaging system 100 processor 110 in accordance with some embodiments of the invention. As shown in FIG. 9, at 902 thermal imaging system 100 processor 110 is configured to acquire a sequence of thermal pixel-level values from one or more locations on a plant 104 from imager 108 based on pixel-level values received from one or more pixels of imager 108 pixel arrays. In some embodiments and as explained elsewhere herein, thermal imaging system 100 processor 110 extracts at 904 for each pixel value one or more of at least thermal variables (a), (b), (c) and (d). In some embodiments, and as explained elsewhere herein, thermal variables (a), (b), (c) and (d) represent plant tissue parameters that effect thermal behavior of the plant tissue (thermal parameters). In some embodiments, thermal imaging system 100 processor 110 displays on display 112 calculated values of at least variables (a), (b), (c) and (d) e.g., in a form of a list.

At 906 processor 110 is configured to calculate a variance value of all the thermal variables associated with each of the locations and at 908 determines plant health status based on the calculated variance.

Alternatively, and optionally, at 910, thermal imaging system 100 processor 110 generates for each pixel, features based on one or more variables extracted at 906. processor 110 is configured to compile at 912, from each of a plurality of pixels within the FOV of imager 108 one or more sets (set (Fa), set (Fb), set (Fc) and set (Fd)) of the features based on one or more of at least variables (a), (b), (c) and (d), and generates at 914 a variance map for each of the compiled sets of features at 912 (VFa, VFb, VFc and VFd) of the at least variables (a), (b), (c) and (d) over the FOV of imager 108.

As explained elsewhere herein, extracted thermal variables (a), (b), (c), (d) and other contributing variables, groups of the same variable or groups of variables from one or more thermal images together with one or a combination of mathematical expressions are analyzed by a computer program product of processor 110, using data mining processes, e.g., to cross-reference data, perform data cleansing, and generate an output in a form of a map indicating and/or identifying various plant health status and/or disease types within the imaged plant imaged area.

In some embodiments, at 914, variance maps for each of the compiled sets at 912 (VFa, VFb, VFc and VFd) of the at least variables (a), (b), (c) and (d) over the FOV of imager 108 are displayed on e.g., display 112 in sequence at 916, or in any combination (e.g., one or more super imposed on each other) at 918, or in any combination and super imposed over a RGB image of imager 108 FOV at 920 to identify unhealthy (unhealthy and/or blemished) plant in accordance with a lookup table based on a predetermined gold standard benchmark, which increases accuracy of the thermal image processing process.

In some embodiments and optionally and as shown in FIG. 9, thermal imaging system 100 processor 110 calculates at 916 cross-section points of one or more data sets e.g., variance data sets (VFa, VFb, VFc and VFd) generated at 908 and identifies at 922 a plurality of pixels sharing close or similar calculated cross-section points. At 924, thermal imaging system 100 processor 110 generates a map corresponding to location of identified pixels from which processing of values obtained results in cross-section points closest to values defined by a predetermined gold standard benchmark and at 926, thermal imaging system 100 processor 110 optionally superimposes the map generated at 924 over an RGB image of imager 108 FOV and at 928 demarcates or identifies unhealthy and/or blemished plant 104 to a sorter.

In some embodiments, cross-section points of one or more data sets e.g., variance data sets (Va, Vb, Vc and Vd) identified by thermal imaging system 100 processor 110 correspond to areas of congruence in overlapping maps of variance data sets (Va, Vb, Vc and Vd). In some embodiments and as explained elsewhere herein, system 100 comprises a source of illumination 114 that illuminates the plant and demarcates unhealthy agricultural product. Alternatively, or additionally and optionally, in some embodiments system 100 comprises a source of ablative energy 116 to mark the unhealthy agricultural product.

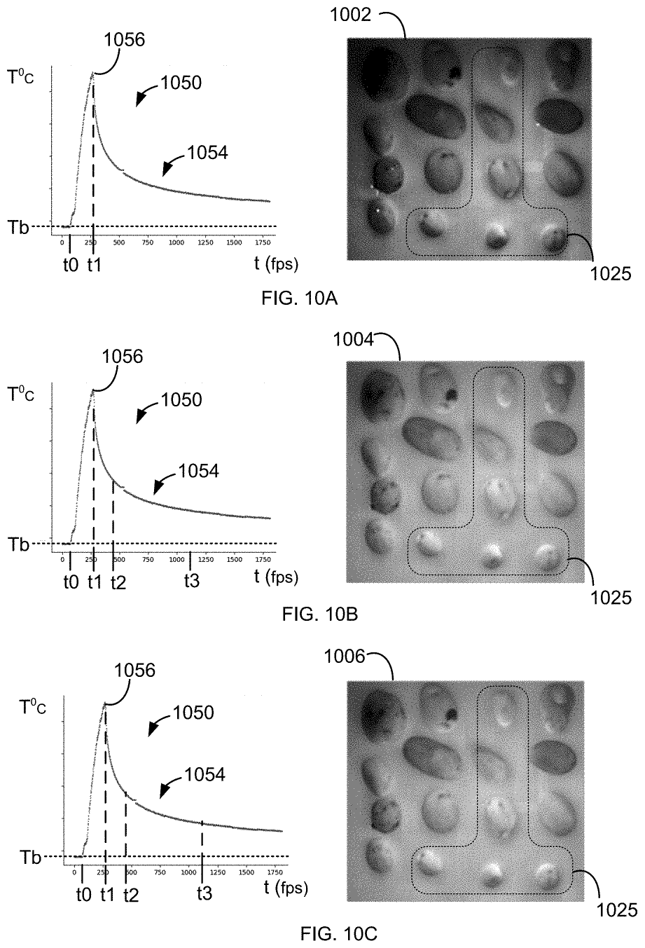

Reference is now made to FIGS. 10A, 10B and 10C, collectively referred to as FIG. 10, in which FIGS. 10A-10C depict exemplary screen thermal images 1000/1002/1004 respectively of potato tubers 1006 on a moving or paused sorting path 106 displayed on system 100 display 112 and a paired thermal graph in accordance with some embodiments of the invention.

As shown in FIG. 10, screen image 1002 includes a thermal map 1025. In some embodiments, system 100 display 112 includes a pixel FOVp indicator 1025 that outlines an area of interest to be examined indicator 1025. In some embodiments, processor 110 forms one or more indicators 1004 that indicate healthy and/or unhealthy plants. In FIG. 10, for example, pixel FOVp indicator 1025 is represented by a frame that represents an area of interest of plants 104 on a moving or paused sorting path 106. In some embodiments, indicator 1025 is controlled, for example, by a joystick, computer mouse or similar control devices. In the exemplary embodiment shown in FIG. 10, pixel FOVp indicator 1025 is placed over a segment of sorting path 106 including one or more plants (potato tubers) 104.

FIG. 10 illustrates an output map generated by a computer program product of processor 110 based on extracted variables (a), (b), (c), (d) and other contributing variables, employing one or more combinations of mathematical expressions described elsewhere herein and used for the generated output maps shown in FIG. 10.

In some embodiments, a method for real-time plants includes actively changing a temperature of at least a portion of an plant from a base temperature (Tb) over a predetermined first period of time (t0 to t1), followed by stopping effecting the temperature change and allowing temperature of the plant to passively return to the base temperature over a second period of time (t1 to t2), while obtaining during said first and second periods of time (t0 to t2) a plurality of digital thermal images of the imaged agricultural product.

In the exemplary embodiment depicted in FIG. 10, thermal images of potatoes 104 have been obtained from three points in time:

Thermal image 1002 was obtained at point of peak temperature 1056 (t1) shown on thermal curve 1050 or shortly after. Active temperature change is ceased at or slightly prior to peak temperature 1056 (t1). Thermal image 1004 was obtained several seconds (t2) beyond point of peak temperature 1056 (t1) on the decay portion 1054 of curve 1050 and thermal image 1006 was obtained further down decay portion 1054 of curve 1050 approximately several seconds (t3) from (t2). In some embodiments, for example, (t1) can be at 10 seconds from initial heating (t0), (t2) between 10 and 50 seconds from (t1) and (t3) over 50 seconds from (t2). In some embodiments, at least three points of temperature measurements obtained from one or more thermal images 1002/1004/1006 are sufficient to extrapolate at least a portion of a thermal graph 1050.

In the exemplary embodiment depicted in FIG. 10, thermal imaging system 100 processor 110 displays on display 112 an output map generated by a computer program product of processor 110. In some embodiments, the generated output map shows calculated variance of values of at least variables (a), (b), (c) and (d) as explained in greater detail elsewhere herein. Additionally, or alternatively, in some embodiments, computer program product of processor 110 receives data contained in thermal images 1002/1004/1006 obtained by imager 108 at, e.g., (t1), (t2) and (t3) and extrapolates a thermal graph 1050 from the thermal images data.

In some embodiments, and as shown in FIG. 10, computer program product of processor 110 is configured to demarcate on display 112 thermal images 1002/1004/1006 healthy and/or unhealthy plants (potatoes) 104 by, for example, displaying an indicator 1025. In the exemplary embodiment depicted in FIG. 10, indicator 1025 demarcates and outlines healthy potatoes within the FOV of imager 108. In some embodiments, and as described elsewhere herein system 100 comprises a source of illumination 114 configured to demarcate the unhealthy plants (e.g., potatoes).

In some embodiments, the method of real-time sorting plants includes processing thermal data contained in the plurality of images that is associated with one or more physiological parameters of the agricultural product, comparing the data with a database of predetermined signature data associated with one or more plant disease types and generating an output indicating identification of plant disease types and/or demarcation of the identified plant infected with the disease or blemished.

In some embodiments, identifying plant disease types includes one or more of tracking changes over period of time in thermal data contained in the thermal images, identifying patterns in said changes and classifying or grouping the patterns of changes into classifications or groups. This is followed by comparing the classified patterns with signature patterns of plant disease types, associating each classification with a database of predetermined signature patterns of plant disease types and identifying plants infected with disease types and/or associating areas within the obtained thermal images with the identified plant disease types.

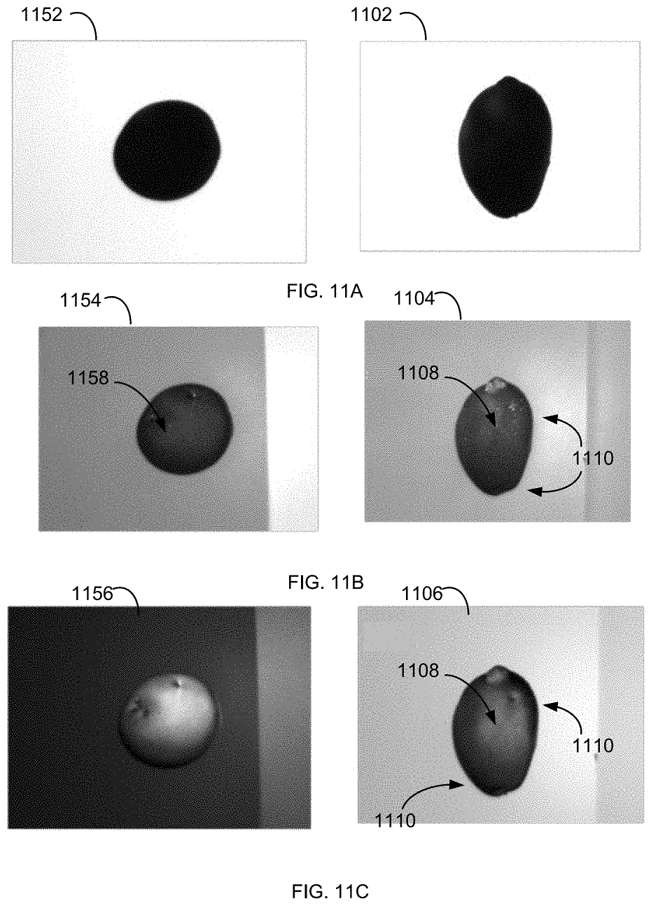

Reference is now made to FIGS. 11A, 11B and 11C, collectively referred to as FIG. 11, which are thermal images comparing a healthy potato and an unhealthy potato in accordance with some embodiments of the invention. In the exemplary embodiment depicted in FIG. 11, screen image 1100 of system 100 display 112 displays frames of imager 108 FOV exhibiting thermal images of a healthy and unhealthy potatoes taken consecutively along a thermal curve (e.g., curves 500 and 600 respectively shown in FIG. 7) that express biothermal behavior of the healthy and unhealthy potatoes in response to active heating and passive cooling over a set period of time (e.g., t0 to t3).

In some embodiments, screen image data represents numerical values calculated by thermal imaging system 100 processor 110 for predetermined variables (a), (b), (c) and (d) as explained elsewhere herein. In some embodiments, thermal imaging system 100 processor 110 is configured to list the variance between calculated numerical values for each of predetermined variables (a), (b), (c) and (d) in the imager 108 FOV display a generated variance map for each isolated value of predetermined variables (a), (b), (c) and (d) within the imager FOV as explained elsewhere herein.

In some embodiments, and as explained elsewhere herein, the generated variance map for each isolated value of predetermined variables (a), (b), (c) and (d) within the imager FOV or any combination thereof is superimposed over a RGB image of the generated variance map for each isolated value of predetermined variables (a), (b), (c) and (d) within the imager FOV so that areas within the generated variance map for each isolated value of predetermined variables (a), (b), (c) and (d) within the imager FOV are identifiable to a naked eye.

In some embodiments, the present disclosure may provide for implementing machine learning algorithms and/or techniques, e.g., for determining a tissue state. In some embodiments, at a training stage, an exemplary machine learning classifier of the present disclosure may be configured to receive, obtain, and/or otherwise having received or obtained a dataset comprising a plurality of tissue thermal parameters, features, and/or variables relating to a plurality of subjects. In some embodiments, these thermal parameters, features, and/or variables are the same or substantially similar to those fully described in detail elsewhere herein.

In some embodiments, a preprocessing stage may include data preparation. Data preparation may include cleaning data, transforming data, and/or selecting subsets of records. In some embodiments, data preparation can include executing pre-processing operations on the data. For example, an imputation algorithm can be executed to generate values for missing data. Up-sampling and/or predictor rank transformation can be executed (e.g., for variable selection) to accommodate class imbalance and non-normality in the data. In some embodiments, executing the imputation algorithm includes interpolating or estimating values for the missing data, such as by generating a distribution of available data for a clinical parameter having missing data, and interpolating values for the missing data based on the distribution.

In some embodiments, a time handling step may be configured to generate a time-dependent representation of one or more parameters, features, and/or variables using, for example, a Fourier transform, polynomial adjustments, decay equations, and/or various statistical tools. In some embodiments, the time handling step may include automatically and/or manually combining a plurality of measurements taken from a subject over a sequence of time periods to determine and/or create a at least one combined parameter and/or feature which may represent patterns of change of the plurality of measurements over time and/or time-series variables.

In some embodiments, a feature extraction step may be configured to generate additional features, e.g., based on relations between existing features in the dataset, and add the additional features to the dataset.

In some embodiments, variable selection may be performed to, e.g., identify the most relevant variables and predictors from the set of obtained parameters. In some embodiments, variable and/or variable selection can include executing supervised machine learning algorithms, such as constraint-based algorithms, constraint-based structure learning algorithms, and/or constraint-based local discovery learning algorithms. In some embodiments, variable selection can be executed to identify a subset of variables in the training data which have desired predictive ability relative to a remainder of the variables in the training data, enabling more efficient and accurate predictions using a model generated based on the selected variables. In some embodiments, variable selection is performed using machine learning algorithms, e.g., Analysis of variance (ANOVA), a boosting ensemble such as XGBoost, Grow-Shrink (“gs”), Incremental Association Markov Blanket (“iamb”), Fast Incremental Association (“fast, iamb”), Max-Min Parents & Children (“mmpc”), or Semi-Interleaved Hiton-PC (“si.hiton.pc”) algorithms. However, various other implementations of such machine learning algorithms may be used to perform variable selection and other processes described herein. In some embodiments, variable selection can search for a smaller dimension set of variables that seek to represent the underlying distribution of the full set of variables, which attempts to increase generalizability to other data sets from the same distribution.

In some embodiments, variable selection may be performed by removing variables that are highly correlated. Several algorithms can be used to search the input dataset with ranked predictors to find a reduced variable set that best represented the underlying distribution of all variables with respect to the infectious complication outcomes. A variable selection filter algorithm can be used to choose the reduced variable set. For example, in some embodiments, one or more of the Maximum Minimum Parents Children (mmpc) and/or the inter-iamb algorithm can be used to choose the nodes of the corresponding Bayesian network as the reduced variable set.

In some embodiments, variable selection is performed to search the training data for a subset of variables which are used as nodes of Bayesian networks. A Bayesian network (e.g., belief network, Bayesian belief network) is a probabilistic model representing a set of variables and their conditional dependencies using a directed acyclic graph. For example, in the context of diagnostic prediction, variable selection can be used to select variables from the training data to be used as nodes of the Bayesian network; given values for the nodes for a specific subject, a prediction of a diagnosis for the subject can then be generated.

In some embodiments, a training dataset for a machine learning classification model of the present disclosure is created, based, at least in part, on the collected parameters and the variable selection process performed as described above. In some embodiments, the training dataset comprises parameters, features, and/or variable sets associated with various tissue states in subjects. The values of the parameters can be received and stored for each of a plurality of subjects. The training dataset can associate the values of the plurality of parameters, features, and/or variable to the corresponding tissue state for each of the plurality of subjects. In some embodiments, the parameters, features, and/or variable sets may be labelled with the corresponding tissue state.

In some embodiments, a machine learning classifier of the present disclosure is trained on the training dataset to generate a classification model. For example, the machine learning classifier can execute classification algorithms (e.g., binary classification algorithms) for each subset of model parameters to generate predictions of tissue state. In some embodiments, the classification algorithms including but not limited to linear discriminant analysis (IDA), classification and regression trees (CART), It-nearest neighbors (KNN), support vector machine (SVM), Gaussian support vector machine (GSVM), logistic regression (GLM), random forest (RF), generalized linear models (GLMNET), and/or naive Bayes (NB). In some embodiments, classification may be defined as the task of generalizing a known structure to be applied to new data. Classification algorithms can include linear discriminant analysis, classification and regression trees/decision tree learning/random forest modeling, nearest neighbor, support vector machine, logistic regression, generalized linear models, Naive Bayesian classification, and neural networks, among others. In some embodiments, a trained machine learning classification model of the present disclosure can include, e.g., cluster analysis, regression (e.g., linear and non-linear), classification, decision analysis, and/or time series analysis, among others. In some embodiments, where variable selection is performed prior to generated the random forest model, the training data is sampled based on the reduced set of variables from variable selection (as opposed to sampling based on all variables).

In some embodiments, after the training stage, a trained machine learning classifier of the present disclosure may be configured to implement a validation process, e.g., through a first evaluation which may include, e.g., a cross-validation. The cross validation may be configured to randomly divide the training set into, e.g., ten folds. The ten-fold validation may then run ten times, for example, using nine different folds of the training set for machine learning modeling, and a tenth fold for validation. The results may be assessed through a computation of statistical measures, e.g., average and a confidence interval of an Area Under a Receiver Operating Characteristic curve (AUROC) for the ten different evaluation folds. In some embodiments, a second evaluation may include an assessment of a machine learning model on a validation set, e.g., the tenth fold for validation which may include 10% of the original data. In some embodiments, a third evaluation may include a statistical analysis, for example, including presenting population characteristics by median and InterQuartile Range (IQR) for skewed data, and a mean with standard deviation for normal distributed data, e.g., using bootstrapping techniques. In some embodiments, a cross validation process of the machine learning model may implement a statistical method configured to estimate a skill of a machine learning model on a limited data sample, e.g., in order to estimate how the machine learning model is expected to perform when used to make predictions on data which was not used when training the machine learning model. In some embodiments, the cross validation process of the machine learning model may include splitting a given data sample into a plurality of groups and/or folds, for example, ten groups and/or folds.

In some embodiments, a trained machine learning classifier fop the present disclosure can be applied, at an inference stage, to a received thermal video stream of a tissue, the generate one or more predictions as to a state of regions within the tissue.

In some embodiments, unsupervised classification models may be employed, using, e.g., to extract parameters, features, and/or variables in an unsupervised manner from thermal image streams of a tissue. In some embodiments, such extracted parameters, features, and/or variables may then be used as an input to the trained machine learning classifier described above.

For example, in some embodiments, thermal images shown in FIG. 11, and optionally displayed on system 100 display 112 are generated by processor 110 following an processing as explained elsewhere herein of raw data extracted from thermal images obtained from imager 118 and are selected by processor 110 from various resulting images to be displayed, being determined to be the most representative and diagnostic images.

In some embodiments, thermal images as depicted in FIG. 11 are taken consecutively during the active heating and passive period of time. Alternatively, and optionally, thermal images as depicted in FIG. 11 are taken continuously by a video thermal imager during the active heating and passive period of time.

### Example 1

In the exemplary embodiments depicted in FIG. 11, thermal images of a healthy potato are compared to thermal images of an unhealthy potato taken at the same times. Images 1102/1104 and 1106 depict a potato infected by Potato Powdery Scab (Spongospora subterranea (Wallr.) Lagerh., f.sp. subterranea Tomlinson). Images 1152/1154 and 1156 depict a healthy potato.

Images 1102/1152 of FIG. 10A were acquired at a pre-heating stage I. Images 1104/1154 of FIG. 10B were acquired several seconds (e.g., between 3 and 5 seconds) after initiation of heating (t0) at the beginning of stage II and images 1106/1156 of FIG. 10C were acquired at stage III immediately after plant temperature peak 506 and the heating (e.g., by heat source 102) was stopped.

As shown in FIG. 11A, both potatoes, healthy potato (image 1152) and unhealthy potato (image 1102) appear the same under ambient temperature and may appear so to a naked eye as well. Once heating begins at stage II, differences are immediately noticed between thermal images 1154 (healthy potato) and 1104 (infected potato) exhibited by a lighter orange color in area 1108 and dark purple margins 1110. Image 1154 (healthy potato) of FIG. 10B also depicts similar dark purple margins however the brighter area 1158 maintains a purple color.

A significant difference is noted once temperature peak (e.g., peak 506) was reached and the heating (e.g., by heat source 102) was stopped (stage III) as imaged in FIG. 10C images 1106 and 1156. The thermal image 1156 of the healthy potato exhibits almost complete loss of the purple coloring and most of the potato exhibits bright yellow coloring. Unlike image 1156, image 1106 exhibiting the unhealthy potato shows the potato to have maintained most of the purple coloring in area 1108 and the deep purple margins 1110.

As explained elsewhere herein, in some embodiments, computer program product of processor 110 is configured to demarcate on display 112 thermal images such as those depicted in FIGS. 10A-10C and demarcate and/or outline healthy potatoes and/or unhealthy potatoes within the FOV of imager 108. In some embodiments, and as described elsewhere herein system 100 comprises a source of illumination 114 configured to demarcate the unhealthy plants (e.g., potatoes).

In some embodiments, the method of real-time sorting plants includes processing thermal data contained in the thermal images that is associated with one or more physiological parameters of the agricultural product, comparing the data with a database of predetermined signature data associated with one or more plant disease types and generating an output indicating identification of plant disease types and/or demarcation of the identified plant infected with the disease types. In this example, computer program product of processor 110 identifies the thermal behavior pattern (change in thermal map or image over time) as a signature thermal map or image of Potato Powdery Scab and displays this diagnosis on an output such as, for example, display 112.

When observing video thermal images of potatoes actively heated and passively cooled the inventors were surprised to notice a point of Rapid Image Change (MC) at which blemishes are noticed instantly. The point of RIC occurs only a few seconds from initiation of active heating (t0). In some embodiments, the MC occurs between 2 and 10 seconds from initiation of active heating (t0). In some embodiments, the MC occurs between 3 and 8 seconds initiation of active heating. In some embodiments, the MC occurs between 4 and 6 seconds from initiation of active heating (t0).

A potential advantage of identification of a point of MC is in that plants with unacceptable blemishes are removed rapidly (within seconds of initiation of the procedure) reducing the number of images needed to be acquired and analyzed to identify unhealthy plants.

### Heat Application Techniques

### Vector Heating

The term “vector heating” as used herein relates to heating along a path that may follow any pattern and not necessarily along a straight line.

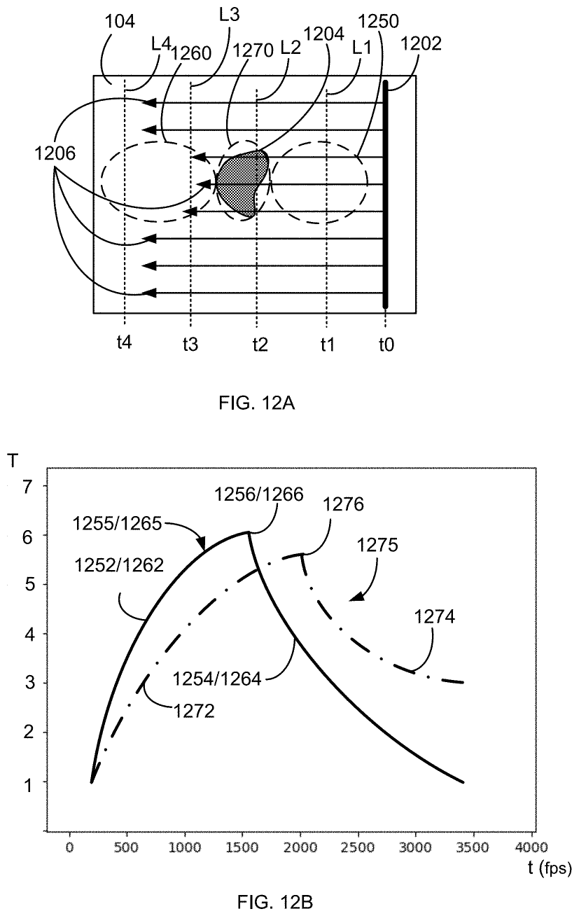

Reference is now made to FIG. 12A, which is a planar view simplified illustration of heat distribution over a portion of an plant in accordance with some embodiments of the invention and 12B, which is a thermal graph of an plant thermal behavior within the portion of the plant in accordance with some embodiments of the invention.

As shown in the exemplary embodiment depicted in FIG. 12A an plant is heated along a line 1202 disposed to one side of a plant 1204. For clarity of explanation, heat distribution from line 1202 in a direction away from the suspected aberrant plant is ignored.

In some embodiments, thermal imaging system 100 is configured to obtain a plurality of thermal images of an FOV of imager 108 over a set period of time (t) and analyze consecutive frames of the plurality of images to extract information regarding variances in thermal parameters of plant tissues over the set period of time.

In some embodiments, thermal imaging system 100 compares speed of heat diffusion through the plant tissues within the FOV of imager 108 in a direction indicated by arrows 1206 over one or more periods of time (e.g., t1, t2, t3, t4) measured from a heat application time (t0). In some embodiments, temperature measurements at periods of time (t1), (t2), (t3) and (t4) are taken along lines (e.g., L1, L2, L3 and L4) parallel to heating line 1202.

As shown in the embodiment depicted in FIG. 12A, during a period of time (t3) the distribution of heat generated by line 1202 is uniform over most of the plant 104 within the FOV of imager 108. However, measurements within an area 1270 delineated by a broken-line circle show a slower diffusion of heat through one or more areas of the plant within the one or more areas as compared to the majority of the surface of plant 104 including delineated areas 1250 and 1260 on either side of area 1270. Thermal imaging system 100 thermal imaging system 100 processor 110 identifies the variance in speed of diffusion through the plant tissues inside area 1270 to be associated with a variance in one or more physiological/thermal parameters associated with the plant within area 1207 and surrounding plant and marks area 1270 as suspected of being aberrant (e.g., unhealthy) e.g., contain blemishes or infections.

Additionally, and optionally, in some embodiments and as explained in greater detail elsewhere herein, thermal imaging system 100 processor 110 is configured to analyze thermal graphs of plant within the FOV of imager 108. As shown in the exemplary embodiment depicted in FIG. 12B, which is a graph of a thermal curve associated with biothermal behavior of heated plant in accordance with some embodiments of the invention, curve 1255/1265 represents thermal curves of suspected areas on a surface of an plant within area 1270, for example, area 1250 and/or area 1260, wherein curve 1275 represents the thermal curve obtained from area 1270 of the surface of an agricultural product. The graphs displayed by thermal imaging system 100 processor 110 show that the overall thermal behavior of plant tissues i.e., response to heating within area 1270 is slower than thermal behavior of plant tissues i.e., response to heating within areas surrounding area 1270 e.g., areas 1250/1260. This is indicated for example, by a shallow growth portion 1272 of curve 1275 in response to heating in respect to a steeper growth portion 1252/1262 of curve 1255/1265. Additionally, and optionally, curve 1275 arrives at peak temperature 1276 later than curve 1255/126, which indicates slower thermal behavior of plant within area 1270. Decay portion 1274 exhibits slower thermal behavior of the plant within area 1270 indicated by a shallow curve in respect to decay portion 1254/1264 of curve 1255/1265 similarly to shallow growth portion 1272.

In some embodiments and as explained in greater detail elsewhere herein, thermal imaging system thermal imaging system 100 processor 110 is configured to analyze the variances exhibited all along thermal behavior curves 1275 and 1255/1265 by processing and compares the graphs as a whole or processing only portions of the curves such as only growing portions 1272 and 1252/1262, only decay portions 1274 and 1254/1264, only by peak temperatures 1276 and 1256/1266 location or any combination thereof and generates a thermal signature derived from the variance between the thermal behavior curves 1275 and 1255/1265 exhibited by the shape of the thermal behavior curve leading to a peak temperature and decaying therefrom and identifies specific plant disease types associated with the thermal signature (e.g., as seen in example 1 disclosed elsewhere herein). In some embodiments, thermal imaging system 100 processor 110 processes information received from at least a portion of an array of pixels as explained in greater detail herein and use the information to indicate an existence of different plant disease types in the examined plant (e.g., normal plant versus unhealthy agricultural product).

In some embodiments, accuracy and specificity of the plant disease type identification can be increased by heating surface of a plant 104 along one or more lines 1202 disposed to one side of the plant 1204.

### Random Spot Heating

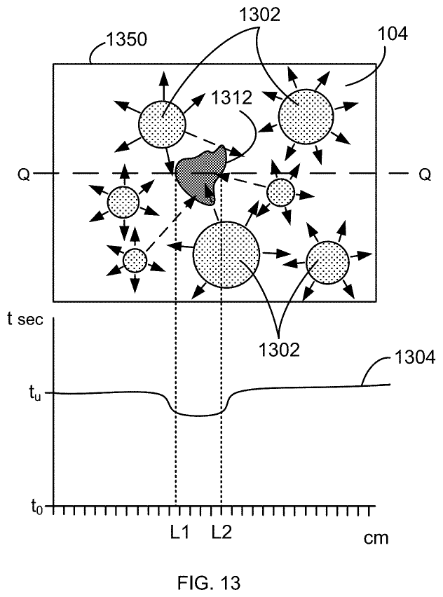

Reference is now made to FIG. 13, which is a graph and a planar view simplified illustration of heat distribution over a portion of a plant in accordance with some embodiments of the invention. In some embodiments and as shown in FIG. 13, a source of heat 102 heats randomly-sized portions 1302 of an of plant 104. In some embodiments, randomly-sized portions 1302 are heated and concurrently and uniformly, e.g., by application of a same level of heat (e.g., Joules) during equal periods of time and the consecutive thermal images at given time intervals are taken by thermal imaging system 100 imager 108.

In some embodiments, and as explained elsewhere herein, thermal imaging system 100 processor 110 processes the obtained images to identify and delineate a plant segment 1312. For example, in some embodiments, processor 110 is configured to analyze and identify a time to temperature uniformity (tᵤ) end point at which a majority (Mc %) of the plant 104 is imaged to be at the same temperature. In some embodiments, a majority (Mc %) of the plant 104 is defined by a percentage of the area of plant 104 within the FOV of imager 108, for example (Mc %) is over 50%, between 50%-99%, 60%-90% and 70%-80%. In some embodiments, processor 110 generates a thermal map 1350 at the end-point (tᵤ) identifying unhealthy or blemished plant segment 1312.

The exemplary graph depicted in FIG. 13 shows a curve 1304 of level of temperature at (tᵤ) along an arbitrary line Q-Q over plant 104. As shown in FIG. 13, graph 1304 exhibits a generally uniform temperature of plant along line Q-Q except for a length between L1 and L2 at which the temperature is lower. In some embodiments, the lower temperature attained by plant along portion L1-L2 of line Q-Q may indicate that the plant comprises a slower growth portion of the thermal curve as explained in detail elsewhere herein identifying the unhealthy or blemished agricultural product. In some embodiments and as described elsewhere herein, thermal imager 100 processor 110 processes consecutive thermal images of the unhealthy or blemished plant expressed by portion L1-L2 of line Q-Q and analyze growth portion of a thermal graph and identify type of plant disease infection or blemish.

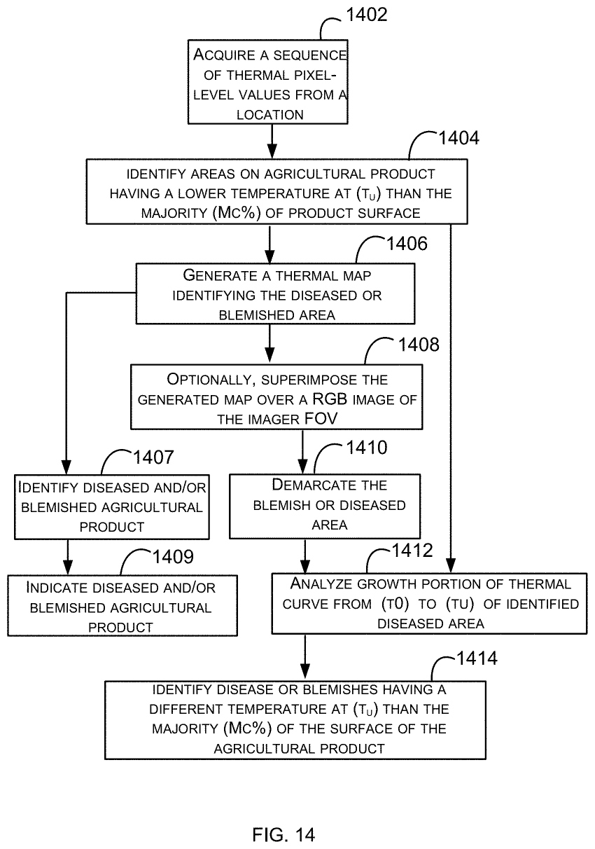

As depicted in FIG. 14, which is an exemplary simplified flow chart illustrating operation of thermal imaging system 100 processor 110 in accordance with some embodiments of the invention, processor 110 is configured to obtain at 1402 thermal images from imager 108 taken over a period of time from (t₀) to (tᵤ) and identifies at 1404 plant tissue groups (e.g., plant surface segment 1412) having a lower temperature at (tᵤ) than the majority (Mc %) of plant 104. At 1406, processor generates a thermal map of plant 104 within the FOV of imager 108 identifying or delineating the unhealthy or blemished plant segment 1412. In some embodiments, at 1407, processor 110 identifies unhealthy and/or blemished plants and at 1409 indicates the unhealthy and/or blemished plants to the sorter. In some embodiments and optionally, at 1408, processor 110 superimposes the map generated at 1406 on a RGB image of plant 104 and at 1410 demarcates the unhealthy or blemished area on the plant 112.

In some embodiments and at 1412, processor 110 is configured to analyze a growth portion of thermal curves of plant tissues identified at 1404 and at 1414 identifies plant disease type infecting at least segment 1412 having a lower temperature at (tᵤ) than the majority (Mc %) of the plant 104.

### Pulsed Heat Application

In some embodiments and as shown in FIGS. 14A and 14B, which are graphs of thermal curves associated with biothermal behavior of heated plant in accordance with some embodiments of the invention, plant 104 is heated by a plurality of pulses of heat over a period of time. In some embodiments, the heat pulses are applied consecutively and uniformly, e.g., by application of a same level of heat (e.g., Joules) during equal periods of time at equal intervals between heating pulses. Thermal imaging system 100 processor 110 is configured to obtain a plurality of consecutive thermal images from imager 108 and analyze the thermal behavior of the plant of plant 104 in response to the heating pulses.

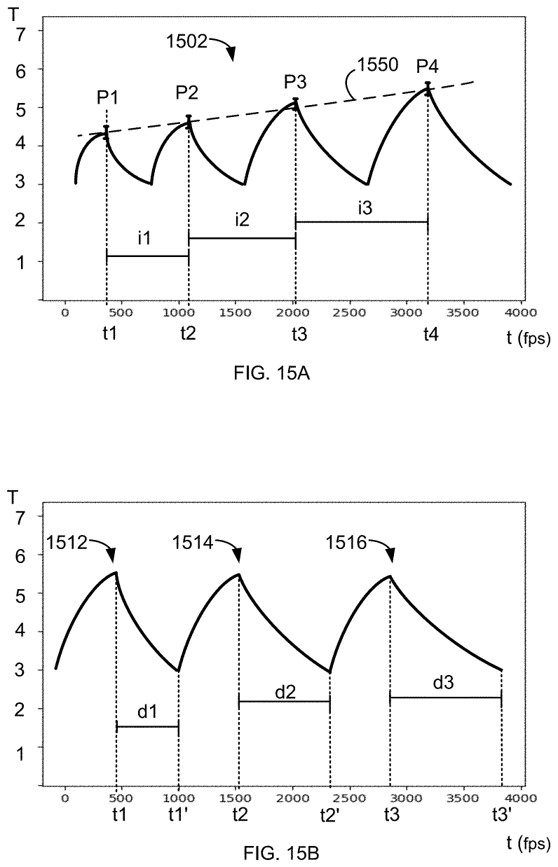

In some embodiments, shown in FIGS. 15A and 15B and explained in greater detail elsewhere herein, different types of plant exhibit different thermal behavior in response to applied pulsed heat, which is expressed by a variance in thermal graphs associated with the thermal behavior. In one example, shown in FIG. 15A, thermal parameters obtained from imaged plant exposed over a period of time to pulsed heat and analyzed by thermal imaging system processor 110 exhibit a curve 1502 comprising one or more growth portions 1504, each followed by one or more decaying portions 1506 and a plurality of temperature peak points 1508.

In some embodiments, processor 110 is configured to perform a top processing on curve 1502 and based on the processing to identify a thermal signature specific to a plant type imaged within an FOVp of a pixel, e.g., by identifying temperature peaks e.g., P1, P2, P3 and P4 of consecutive curves in response to consecutive heat pulses at given times e.g., t1, t2, t3 and t4 and processing the relationship between the peaks e.g., time intervals between the peaks e.g., i1, i2, i3 and i4 or a growing linear regression 1550 of the calculated peaks.

In some embodiments, thermal imaging system 100 processor 110 is configured to execute a comparative processing on selected portions only of the thermal curve e.g., a growth portion, a decay portion and/or a peak temperature at the meeting point of the growth portion and the decay portion e.g., the exemplary graph shown in FIG. 15B, exhibits a growing decay periods d1 between t1 and t1′, d2 between t2 and t2′ and d3 between t3 and t3′ of curves 1512, 1514 and 1516 in response to consecutive heat pulses. In some embodiments, processor 110 is configured, based on the processing to identify a thermal signature specific to a plant type imaged within an FOVp of a pixel, e.g., by identifying a thermal behavior pattern specific to a plant disease type.

### Fractional Heating

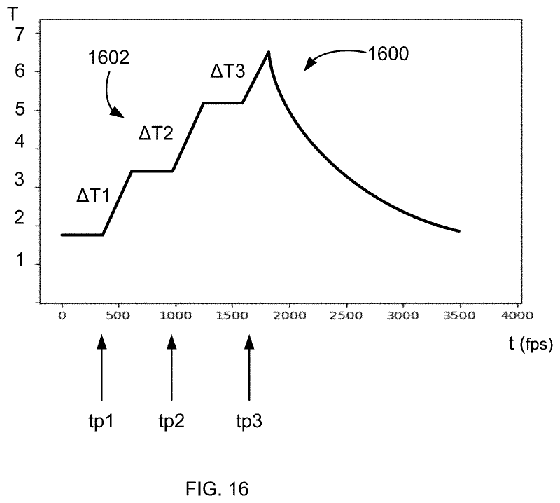

In some embodiments and as shown in FIG. 16, which is a graph of a thermal curve associated with biothermal behavior of heated plant in accordance with some embodiments of the invention, plant 104 is heated fractionally. In some embodiments, heat is applied by a plurality of pulses of set (e.g., same level of heat or Joules) at predetermined intervals, e.g., equal or varying in length and thermal imaging system 100 imager 108 obtains consecutive thermal images throughout the growth portion 1602 of the obtained thermal curve 1600.

In the exemplary embodiment depicted in FIG. 16, three heat pulses are applied at three points in time—tP1, tP2 and tP3 resulting in a stepped growth portion 1602 of curve 1600 having three fractions ΔT1, ΔT2 and ΔT3. A potential advantage of fractional heating is in that the processing is carried out on fractions of growth portion 1602 rather than on the full growth portion 1600 allowing for increased resolution and accuracy of the aberrant plant tissue identification. In some embodiments, variance between plant disease types is expressed in variances within only one fraction of fractions ΔT1, ΔT2 and ΔT3 providing a higher resolution of an agricultural product-type signature pattern and increasing the accuracy and specificity of the plant type identification.

### 3D Heating

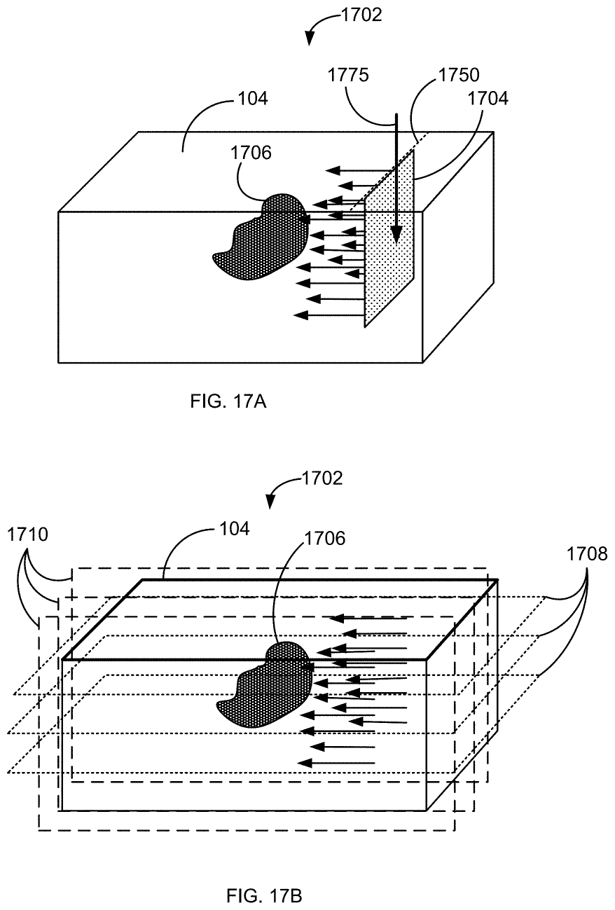

Reference is now made to FIGS. 17A and 17B, which are sectional view simplified illustrations of heat distribution inside a portion of a plant in accordance with some embodiments of the invention. In some embodiments, a volume of plant 1702 under plant 104 is heated along a plane 1704 using three-dimensional heating systems such as Ultrasound, Laser, IR or RF radiation applied at varying frequencies along a line 1750 disposed to one side of a suspected aberrant plant 1706 in a direction from the surface into deeper plant indicated by an arrow 1775.

As shown in FIGS. 17A and 17B heat distribution inside a portion 1702 under plant 104 occurs along lines 1708. For clarity of explanation, heat distribution from plane 1704 in a direction away from the suspected plant segment 1706 is ignored.

In some embodiments, imaging system 100 processor 110 is configured to process and analyze a plurality of thermal images taken by a 3D thermal imaging system e.g., MRI, CT Scanner, Ultrasound transceiver, RF transceiver or similar, concurrently or consecutively along one or more planes at varying spatial orientation in respect to plant 104. In the exemplary embodiment illustrated in FIG. 17, a plurality of thermal images taken by a 3D thermal imaging system, concurrently or consecutively are taken along a plurality planes spatially orientated parallel (planes 1708) and/or perpendicular (planes 1710) in respect to plant 104.

As shown in FIG. 17B and in some embodiments, thermal imaging system 100 processor 110 is configured to compile the thermal behavior data obtained from thermal images taken along plurality of planes 1708 and/or planes 1710 and as explained in greater detail elsewhere herein, extract information regarding one or more physiological thermal parameters associated with plant identified as aberrant plant tissues 1706 in one or more obtained thermal images and generate at least a three-dimensional outline of suspected aberrant plant 1706.

In some embodiments, the duplicity of at least part of the data obtained by thermal imaging system 100 processor 110 and comparison between obtained data from the obtained images increases accuracy and specificity of the plant disease type identification and location inside plant under plant 104. In some embodiments, thermal imaging system 100 processor 110 is configured to superimpose the 3D outline of suspected aberrant plant 1706 onto a RGB 3D image of plant under surface of plant 104 to assist the sorter clearly and accurately identify the borders of suspicious areas 904 within the agricultural product.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

Methods and computer program products are disclosed herein that may automatically construct (i.e., without human intervention) a list of relevant claims and supportive evidence given a topic under consideration (TUC). Thus, for example, one may extract persuasive claims supporting his or her point of view as well as be prepared for counter claims which the other side may raise while discussing the TUC.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method comprising:
receiving thermal data associated with a region of a plant, wherein said thermal data is sampled over time while said plant is being thermally disturbed;
deriving from said thermal data a set of features with respect to each of a plurality of locations within said region;
comparing each of said sets of features with a set of known sets of features, each associated with a specified health state of said plant; and
determining a health state of said plant based on said comparing.

2. (canceled)

3. The method according to claim 1, wherein said thermal data is received from at least one of thermal imaging, infrared (IR) sensor, mercury thermometer, resistance thermometer, thermistor, thermocouple, semiconductor-based temperature sensor, pyrometer, gas thermometer, laser thermometer and ultrasound.

4. The method according to claim 1, wherein said thermal data is received by thermal imaging, and wherein each of said locations comprises a pixel or a voxel of an image.

5. The method according to claim 1, wherein said set of features comprises at least one feature based on at least one of ambient temperature and a heat source temperature.

6. The method according to claim 1, wherein said thermal disturbing comprises at least one of: actively effecting a change in temperature in at least a portion of said tissue from an initial temperature to an end temperature, actively effecting a change in temperature in at least a portion of said tissue for a specified period of time, passively allowing a change in temperature in at least a portion of said tissue from an initial temperature to an end temperature, and passively allowing a change in temperature in at least a portion of said tissue for a specified period of time.

7. (canceled)

8. (canceled)

9. (canceled)

10. The method according to claim 1, wherein said determining is performed by a machine learning classifier trained, at a training stage, on a training set comprising:
a) a plurality of sets of features, each derived from thermal data sampled at each of a plurality of locations within a plurality of plants, while said plants are being thermally disturbed; and
b) labels associated with a health state of each of said locations.

11. The method of claim 10, further comprising applying, at an inference stage, said trained machine learning classifier to at least one target set of features derived from thermal data sampled at a location of a plant, while said plant is being thermally disturbed, to determine a health state of said plant location.

12. A computer program product comprising a non-transitory computer-readable storage medium having program code embodied therewith, the program code executable by at least one hardware processor to:
receive thermal data associated with a region of a plant, wherein said thermal data is sampled over time while said plant is being thermally disturbed;
derive from said thermal data a set of features with respect to each of a plurality of locations within said region;
compare each of said sets of features with a set of known sets of features, each associated with a specified health state of said plant; and
determine a health state of said plant based on said comparing.

13. (canceled)

14. The computer program product according to claim 12, wherein said thermal data is received from at least one of thermal imaging, infrared (IR) sensor, mercury thermometer, resistance thermometer, thermistor, thermocouple, semiconductor-based temperature sensor, pyrometer, gas thermometer, laser thermometer and ultrasound.

15. The computer program product according to claim 14, wherein said thermal data is received by thermal imaging and wherein each of said locations comprises a pixel or a voxel.

16. The computer program product according to claim 12, wherein said thermal disturbing comprises at least one of: actively effecting a change in temperature in at least a portion of said tissue from an initial temperature to an end temperature, actively effecting a change in temperature in at least a portion of said tissue for a specified period of time, passively allowing a change in temperature in at least a portion of said tissue from an initial temperature to an end temperature, and passively allowing a change in temperature in at least a portion of said tissue for a specified period of time.

17. (canceled)

18. The computer program product according to claim 12, wherein said set of features comprises at least one feature based on at least one intrinsic plant thermal parameter affecting thermal behavior of the plant cell.

19. (canceled)

20. (canceled)

21. (canceled)

22. (canceled)

23. A system, comprising:
a thermal sensor configured to sample a thermal data associated with a region of a plant, while said plant is being thermally disturbed; and
a processor configured to:
receive said thermal data;
derive from said thermal data a set of features with respect to each of a plurality of locations within said region;
compare each of said sets of features with a set of known sets of features, each associated with a specified health state of said plant; and
determine a health state of said plant based on said comparing.

24. The system according to claim 23, wherein said system comprises a heating or cooling source directed said region of said plant and configured to actively heat or cool said plant.

25. (canceled)

26. The system according to claim 23, wherein said thermal data is received from at least one of thermal imaging, infrared (IR) sensor, mercury thermometer, resistance thermometer, thermistor, thermocouple, semiconductor-based temperature sensor, pyrometer, gas thermometer, laser thermometer and ultrasound.

27. The system according to claim 23, wherein said thermal data is received by thermal imaging and wherein each of said locations comprises a pixel or a voxel.

28. The system according to claim 23, wherein said thermal disturbing comprises at least one of: actively effecting a change in temperature in at least a portion of said tissue from an initial temperature to an end temperature, actively effecting a change in temperature in at least a portion of said tissue for a specified period of time, passively allowing a change in temperature in at least a portion of said tissue from an initial temperature to an end temperature, and passively allowing a change in temperature in at least a portion of said tissue for a specified period of time.

29. (canceled)

30. The system according to claim 23, wherein said set of features comprises at least one feature based at least one intrinsic plant thermal parameter affecting thermal behavior of the plant cell.

31. (canceled)

32. (canceled)

33. (canceled)
